(19) United States
(12) Patent Application Publication
(10) Pub. No.: US 2005/0232933 A1
(43) Pub. Date: Oct. 20, 2005

(54) CMV-IE1 peptides and method of use

(76) Inventors: John A. Zaia, Arcadia, CA (US); Ghislaine Gallez-Hawkins, La Verne, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/953,769

(22) Filed: Sep. 30, 2004

Related U.S. Application Data
(60) Provisional application No. 60/506,734, filed on Sep. 30, 2003.

(51) Int. Cl. A61K 48/00; A61K 39/12; C07K 14/005

(52) U.S. Cl. 424/186.1; 530/350; 514/44

(57) ABSTRACT

IE1 peptide antigens that are recognized by and stimulate production of CMV-specific cytotoxic T lymphocytes are useful for vaccines, in the form of peptides, DNA vaccines or cellular vaccines, and also for diagnostic methods.

### CROSS REFERENCE TO RELATED APPLICATION

This application claims priority from U.S. Provisional Application No. 60/506,734, filed Sep. 30, 2003, the disclosures of which are hereby incorporated by reference in their entirety.

### STATEMENT REGARDING FEDERALLY SPONSORED RESEARCH

This invention was made with Government support in the form of Grant No. P01 CA30206, from the United States Department of Health and Human Services, National Cancer Institute. The United States Government may have certain rights in this invention.

### BACKGROUND OF THE INVENTION

1. Technical Field

This invention relates to the field of immunology. Human cytomegalovirus IE1 peptide antigens recognized by and stimulating production of CMV-specific CTL form part of this invention, as well as methods for using the antigenic peptides to produce antigen presenting cells and CMV-reactive cytotoxic T lymphocytes and for manufacture of vaccines and diagnostic reagents. DNA constructs encoding the antigenic peptides also are contemplated for use, for example, in DNA vaccines.

2. Background Information

Developing a CMV vaccine remains an important focus of research in immunosuppressed patients and in CMV seropositive mothers with young children. Adler et al., J. Infect. Dis. 171:26-32, 1995; Plotkin et al., Science 265:1383-1385, 1994. Murine models to predict human CMV vaccine immunogenicity are being developed to detect the presence of specific immune responses, such as cytolytic T lymphocyte (CTL) function, CD4⁺ IFN-γ helper response or antibody response to specific CMV proteins. Berencsi et al., J. Gen. Virol. 174:2507-2512, 1993; Del Val et al., J. Virol. 65:3641-3646, 1991; Villacres et al., Virology 270:54-64, 2000; Kern et al., Eur. J. Immunol. 130:1676-1682, 2000; Price et al., Immunology 78:14-21, 1993; Morello et al., J. Virol. 176:4822-4835, 2002; Pande et al., Scand. J. Infect. Dis. Suppl. 99:117-120, 1995; Pepperl et al., J. Virol. 74(13):6132-6146, 2000.

The use of the transgenic A2/Kb mouse containing the human HLA A*0201 with the murine alpha3 chain (Kb) also has been of interest. Engelhard et al., J. Immunol. 146:1226-1232, 1991; Newberg et al., J. Immunol. 149:136-142, 1992; Vitiello et al., J. Exp. Med. 173:1007-1015, 1991. When immunized with human CMV DNA, these well-known model mice develop murine CTL that recognize human class I MHC restricted peptides on antigen presenting cells (APC) in a manner which correlates with human responses. Transgenic A2/Kb mice have become a useful model to investigate peptide recognition of specific proteins, with results that can be extrapolated to human subjects.

The success of vaccine strategy for CMV typically is evaluated by detection of CTL activity processed through the class I pathway. Although in vitro stimulation (IVS) of CTL can be achieved using various cell lines infected with CMV or with recombinant viruses expressing proteins of interest such as pp65 or IE1, the accuracy and sensitivity of IVS assays is enhanced when peptides that bind specifically to the MHC molecule of the APC are used. The immunodominant HLA A*0201-restricted peptide of CMV pp65 (pp65₄₉₅₋₅₀₃) has been studied, however there is no recognized immunodominant peptide for the CMV IE1 protein, despite the presence of a dominant IE1-specific CTL response. Boppana et al., Virology 222:293-296, 1996; Diamond et al., Blood 90: 1751-1767, 1997; Gallez-Hawkins et al., Scand. J. Immunol. 55:592-598, 2002; Wins et al., J. Virol. 70:7569-7579, 1996; Frankenberg et al., Virology 295:208-216, 2002; Gyulai et al., J. Infect. Dis. 181:1537-1546, 2000; Kern et al., J. Virol. 173:8179-8184, 1999; Khan et al., J. Infect. Dis. 185:1025-1034, 2002. Reports have described the stimulatory effect of peptides IE1ₚ₃₁₅₋₃₂₃, IE1ₚ₃₁₆₋₃₂₄ and IE1ₚ₃₅₄₋₃₆₂ from CMV IE1 in the context of HLA A*0201 in an intracellular cytokine (ICC) or CTL assay. Frankenberg et al., Virology 295:208-216, 2002; Gyulai et al., J. Infect. Dis. 181:1537-1546, 2000; Khan et al., J. Infect. Dis. 185:1025-1034, 2002; Retiere et al., J. Virol. 74:3948-3952, 2000. However not all CMV-seropositive subjects respond to these peptides. Khan et al., J. Infect. Dis. 185:1025-1034, 2002.

Current understanding of the protein-specific immune response to CMV is based upon studies on CMV pp65, the abundant tegument protein of the virus which is present at the time of first infection, and CMV immediate early protein (IE1) which is expressed during the initial viral transcription. The immune response targeted to the CMVpp65 protein has been well described in HCT recipients. The pp65-495 peptide (NLVPMVATV; SEQ ID NO:10) was shown to induce a CMVpp65 protein specific intracellular cytokine response in HLA A*0201 persons. When this peptide was used to fold tetrameric MHC molecules for detection of specific CMV CD8 cells, up to 20% pp65-CMV-specific cells were enumerated. The robust cellular immune response to CMVpp65 therefore has been used to characterize immune response to CMV in other HLA contexts such as B7, A1, B8 and B35.

Investigation of the immune response to cytomegalovirus is important for identifying methods of protection from and treatment for CMV disease. Approaches used to look for IE1-specific epitopes include scanning epitope libraries for IFN-γ or TNF-α ICC (TNF-α intracellular cytokine) expression in PBLs and multiple IVS to generate human CTL cell lines. See Kern et al., J. Virol. 173:8179-8184, 1999; Retiere et al., J. Virol. 74:3948-3952, 2000; Frankenberg et al., Virology 295:208-216, 2002. Yet, to date, a definitive description of which IE1 peptides contribute to the stimulation of CD8 cells in the context of HLA A*0201 molecule is lacking in the art.

Therefore, there exists a need in the art for methods which can identify immunoreactive peptides of viral proteins, particularly of HCMV and of its IE1 gene. Peptides identified as immunoreactive are useful for vaccines as well as diagnostic reagents. Vaccine peptides from viral proteins may be used for enhancing the immune system with respect to the virus in seropositive and seronegative individuals.

### SUMMARY OF THE INVENTION

Accordingly, this invention provides the compositions comprising peptides ILDEERDKV (SEQ ID NO:1), TMYGGISLL (SEQ ID NO:2) and VLEETSVML (SEQ ID NO:3). Vaccine compositions, including peptide vaccines, DNA vaccines and cellular vaccines comprising, encoding and presenting these peptides also are provided. The invention also relates to methods for stimulating CMV-specific cytotoxic T-lymphocytes by administering such peptides and vaccines, and methods for diagnosing the presence of CMV-specific cytotoxic T-lymphocytes in a patient sample by contacting the sample in vitro with a reagent comprising these peptides, including tetramer reagents and antigen presenting cell reagents.

One embodiment of the invention provides a CMV peptide composition which comprises a peptide of SEQ ID No: 1, 2 or 3, which may be a vaccine or a diagnostic reagent. Another embodiment provides a peptide of SEQ ID No: 1, 2 or 3. Additional embodiments provide an antigen presenting cells that presents these peptides. Further embodiments provide methods of stimulating the production of CV-specific cytotoxic T lymphocytes in a patient or diagnosing the presence of CMV-specific cytotoxic T lymphocytes in a patient sample using the compositions discussed above.

### BRIEF DESCRIPTION OF THE FIGURES

FIG. 1 is a map of a recombinant AAV plasmid with the internal expression cassette removed from CWRSP and replaced by the pcDNA3.1+ cassette, leaving the ITR from AAV2 intact. The CMV genes, IE1 and pp65mII, were inserted into the multi-cloning site.

FIG. 2 shows peak fluorescence results for B7 peptide (white area)- and IE1-81 peptide (black area)-incubated T2 cells.

FIG. 3 shows peak fluorescence results for B7 peptide (white area)- and IE1-297 peptide (black area)-incubated T2 cells.

FIG. 4 provides results for CTL killing mediated by immune splenocytes on T2 (panels A, B and C) or A293 (panel D) target cells. Splenocytes were obtained from mice (M1-M12) immunized as indicated in Example 3.

FIG. 5 provides target cell lysis data by splenocytes from mice immunized with pcDNA-IE1 and GM-CSF that responded to T2 target cells loaded with each individual IE1 peptide.

FIG. 6 shows IE1 CTL responses of splenocytes from HHDII mice immunized with recAAV-IE1 and stimulated with autologous blasts loaded with a mix of IE1 peptides. Panel A: recognition of endogenously processed IE1 peptides; Panel B: recognition of IE1 peptide mixture; Panel C: recognition of individual IE1 peptides, as indicated. Panels D-F present the same information for A2/Kb mice.

FIG. 7 is a photograph showing cells stained for IE1 expression 4 and 18 hours after transduction into rAAV-IE. FIGS. 7A and 7C show cells treated with 10 μM cycloheximide. FIGS. 7B and 7D show a control transduction without cycloheximide.

FIG. 8 depicts percent chromium release in assays at the indicated effector-target ratios using T2 cells loaded with CMV-pp65-495 (8A) and LCLA2 cells loaded with the same peptide (8B). Mice M1-M8 were subjected to a prime-boost regimen using low dose rAAV-pp65mII. Mice M9-M10 received control vectors without CMV genes.

FIG. 9 shows percent chromium release in assays at the indicated effector-target ratios by cells from A2/kb mice inoculated with pcDNAintIE1, pcDNAintpp65mII and pcDNAintgm-CSF and then boosted with rAAV-IE1 and rAAV-pp65mII. Control mice M9 and M10 received vector only. FIG. 9A: percent lysis after one in vitro stimulation with IE1-mix, T2 cells loaded with CM-IE1 mix. FIG. 9B: percent lysis after one in vitro stimulation with CMV-pp65₄₉₅₋₅₀₃, T2 cells loaded into CMV-pp65₄₉₅₋₅₀₃. FIGS. 9C and 9D show the same data, respectively, after two in vitro stimulations.

FIG. 10 shows results of an ELISA of sera from A2/kb mice (M1-M4), HHDII mice (M5-M7) and control mice. Mice M1-M7 were immunized with pcDNAintIE1, pcDNAintpp65mII and pcDNAintgm-CSF, followed 30 days later with an inoculation of rAAV-IE1 and rAAV-pc65mII. FIG. 10A shows results for IE1 antibodies; FIG. 10B shows results for pp65 antibodies.

FIG. 11 shows results of tetramer binding assays using HLA A2 folded with pp65-495 (11A), IE1-297 (11B) or no tetramer (11C), at time of harvest.

FIG. 12 shows results of a tetramer assay using HLA A2 folded with pp65-495 (12A), IE1-297 (12B) or no tetramer (12C), after 6 days stimulation with the respective peptide.

FIG. 13 shows ICC/IFN-γ+ positive samples as a percentage of all samples in the CMV reactivation and the No CMV groups after overnight stimulation with the indicated CMV peptides. The detection limit was 0.01% of CD8+/IFN-γ⁺ cells or 1×10⁵ cells/L.

FIG. 14 provides data showing levels of CD8+/IFN-γ⁺ cells over time after HCT in the CMV reactivation group (FIGS. 14A and 14B) and in the No CMV group (FIGS. 14C and 14D). FIGS. 14A and 14C were stimulated with pp65-495; FIGS. 14B and 14D show the sum of cells stimulated with IE1-256, IE1-297 and IE1-316. The number of IFN-γ+ cells is expressed as 1×10⁵ cells/L. The median value is calculated at each time point and shown on the graph as a continuous line.

FIG. 15 shows CMV reactivation in relation to appearance of CMV immune cells by ICC in time post-HCT for each individual (#54, #70, #93, #94, and #105) as time of PCR positivity post-HCT in relation with pp65-495 stimulation (FIGS. 15A through 15E), and with IE1-256, IE1-297 and IE1-316 stimulation (FIGS. 15F through 15J). The number of IFN-γ⁺ cells is expressed in units of 1×10⁵ cells/L (Y axis). Time after HCT for each subject is indicated on the X axis. When available, the number of cells is shown for the donor before and after G-CSF treatment (preG and postG).

FIG. 16 shows the time course after HCT of the number of tetramer positive cells specific to pp65-495 (FIGS. 16A and 16A) and the sum of tetramer positive cells specific to IE1-256, IE1-297 and IE1-316 (FIGS. 16B and 16D). FIGS. 16A and 16B represent the CMV reactivation group, and FIGS. 16C and 16D represent the No CMV group. The number of IFN-γ⁺ cells is expressed in units of 1×10⁵ cells/L and the median value is calculated at each time point and shown on the graph as a continuous line.

### DESCRIPTION OF THE PREFERRED EMBODIMENTS

A2/Kb transgenic mice may be made as described by Benmohamed et al., Hum. Immunol. 61:764-779, 2000 by microinjection of a chimeric molecule containing the α1 and α2 domains of the HLA A*0201 gene and the α3 domain of the murine H-2Kb into fertilized eggs from C57BL/6 mice. These mice thus contain the human HLA A*0201 Kb molecule in the C57BL/6 background. The transgenic mouse strain HHDII expresses a transgenic monochain histocompatibility class I molecule in which the C terminus of the human β2m is covalently linked to the N terminus of a chimeric heavy chain (HLA-A-0201-α1, -α2, H-2Db -α3-transmembrane, and intracytoplasmic domains). These mice are described in Firat et al., Eur. J. Immunol. 29(10):3112-3121, 1999, the disclosures of which are hereby incorporated by reference.

T2 cells, ATCC accession no. CRL-1992, are defective for endogenous class I presentation, but peptide binding to the MHC molecule stabilizes its expression on the cell surface. The stabilized MHC molecule can be detected by flow cytometry with a monoclonal antibody to the HLA A*0201 molecule. T2 cells express HLA A*0201 but not the HLA DR and are class II MHC antigen negative. They have been used extensively as target cells when loaded with peptides that bind to the MHC A*0201 molecule. A293 cells are human embryonic kidney cells. A293-IE1 is a stably transfected human kidney cell line endogenously expressing the CMV-IE1 protein. All cells used in experiments were mycoplasma-free and were maintained in RPMI-1640 supplemented with 10% FBS, penicillin-streptomycin and 2 mM glutamine.

To identify IE1 peptides recognized in vivo during a naturally-acquired HCMV infection, transgenic HLA A*0201 mice were immunized with DNA encoding CMV IE1. Immunized splenic lymphocytes from the mice were stimulated in vitro using a pool of 5 IE1 nonapeptides chosen based on their likelihood (through conformance to a motif) to be HLA A*0201-restricted targets of CTL. The peptide IE1ₚ₃₁₆₋₃₂₄ (IE1-316; SEQ ID NO:3; see Table II, below) was identified as a potential epitope recognized in vivo. In addition, a transgenic mouse system revealed a robust specific CTL response recognizing IE1ₚ₂₉₇₋₃₀₄ (IE1-297; SEQ ID NO:2; See Table II, below). The immunoreactivity of IE1ₚ₂₉₇₋₃₀₄ was confirmed by testing peripheral blood mononuclear leukocytes (PBML) from CMV seropositive human blood for the presence of CD8⁺ cells reactive to this peptide. Among the IE1 peptides which are presented in an HLA A*0201-restricted manner, this study recognized IE1ₚ₂₉₇₋₃₀₄ as an important, previously unrecognized CD8 epitope of CMV. IE1ₚ₂₉₇₋₃₀₄ triggers a CD8⁺ cell response to CMV IE1 in immunosuppressed HLA A*0201 subjects as determined by IFN-γ production. This strategy can be used to identify immunoreactive peptides of viral proteins and may be useful to further characterize and fine map the immune response to CMV.

CMV-specific immune responses can be quantitatively measured by fluorescence activated cell sorting (FACS) using either the HLA-peptide-specific tetramer binding assay (tet+) or measurements of intracellular cytokine (ICC assay and ELISPOT test) in response to antigen. The risk for CMV disease, therefore, should be definable using such quantitative immunologic assays since hematopoietic stem cell transplant (HCT) recipients with a critical level of CMV-specific CD8/tet+ cells per liter are no longer at risk for CMV complications. CMV encodes more than 200 polypeptides. The immune response to CMV is complex and involves multiple protein targets, but for only a few of these antigens do we know the peptide-specific recognition sites.

The IE1-297 epitope was recognized in A2/Kb mice following immunization with DNA expressing CMV IE1. Recognition of IE1-256 was sporadic. The effectors from A2/Kb mice lysed only target cells presenting the IE1-297 epitope. Human CD8 cells from 2 out of 4 CMV seropositive subjects secreted IFN-γ following stimulation with IE1-297. These results show that IE1-297 peptide can be used in assays to detect immune responses in human cells. IE1-297 is a strong stimulator of CMV CTL responses in a well-known transgenic mouse model known to correlate to human responses. A response specific to IE1-297 also was confirmed in human CD8 cells. Therefore, a pool of selected CMV-IE1 peptides can be used in IFN-γ or TNFα-ICC assays able to detect CMV-IE1 reactivity in all HLA A*0201 subjects.

Using particular individual IE1 peptides as immunogens whether with a DNA or a viral vector approach, does not take into account the mutations that occur naturally in the CMV IE1 wild type. These mutations may contribute to the low incidence of response to individual IE1 peptides. Table I reports the IE1 peptides that have been studied to date. The locations of putative mutations are underlined. See Frankenberg et al., Virology 295: 208-216, 2002. Typically, the mutations that would most dramatically affect the binding of an epitope into the MHC molecule are situated at the anchor site, amino acid positions p2 and p9 of a peptide. See Falk et al., Semin. Immunol. 15:81-94, 1993; Zaia et al., J. Virol. 75:2472-2474, 2001. The only peptide showing a p2-p9 unstable mutation site is IE1-315, first described by Retiere (J. Virol. 74:3948-3952, 2000) using a TNF release assay. IE1-315 has been reported as stimulatory by elispot in only one subject out of 18. See Khan et al., J. Infect. Dis. 185:1025-1034, 2002. However IE1-316, which contains possible mutations at site p1 and p8, was more frequently recognized in this same study (6 out of 18) in an IFN-γ elispot assay. IE1-297, possibly mutated at p1 and p5, is a strong candidate for CMV immune recognition as well, since 3 of 4 patient PBLs responded in an ICC (intracellular cytokine) assay. Therefore, despite the presence of point mutation sites in IE1 epitopes, provided the anchor sites are intact, IE1-297 and IE1-316 peptides are presented by the MHC molecule after immunization with or exposure to CMV in HLA A*0201 subjects. In summary, IE1-297, IE1-316 and IE1-256 are peptides that stimulate CD8 human cells (Table I).

TABLE I

Summary of Stimulatory CMV IE1 HLA A *0201

Restricted Peptides.

| | | | | | |
|---|---|---|---|---|---|
| | | | | | SEQ ID |
| A | B | C | D | NO: | |

| | | | | | | |
|---|---|---|---|---|---|---|
| IE1-81 | VLAELVKQI | neg | neg | (p2) neg | | 4 |
| IE1-256 | ILDEERDKV | 1+ | neg | (p3) neg | | 1 |
| IE1-297 | TMYGGISLL_{C} | 3+ | neg | (p1) neg | | 2 |
| IE1-303 | SLLSEFCRV | | neg | (p9) poor | | 5 |
| IE1-304 | LLSEFCRVL | neg | neg | (p4) neg | | 6 |
| IE1-315 | YVLEETSVM | | 1+ | | 1+ | 7 |
| IE1-316 | VLEETSVML | 2+ | 3+ | | | 3 |
| IE1-354 | YILGADPLRV | | | (p6) 3+ | | 8 |
| IE1-355 | ILGADPLRV | | neg | (p10) poor | | 9 |

A: Present Report
B: Khan et al. 2002, JID; 185:1025-1034 (ELISPOT).
C: Frankenberg at al. 2002, Virology; 295:208-216 (IVS4).
D: Retiere at al. 2000, J Virol.; 74:3948-3952 (TNF release).
Underlining represents published area of amino acid mutations in the TE1 gene.

Using the methods outlined here, peptides suitable for vaccine and diagnostic purposes can be identified. A cocktail peptide vaccine with epitopes recognized by individuals having a variety of haplotypes may be useful to vaccinate large multi-ethnic populations. If a vaccine has broad enough reactivity to be useful for at least 80% and preferably 90% or 95% of most ethnic populations, it is more suitable for public health.

For CMV IE1, there is no dominant CMV-IE1 peptide. An IE1-316 (VLEETSVML; SEQ ID NO:3)-specific peptide response has been reported in cells from 6 of 18 subjects using an ELISPOT assay. The IE1-315 peptide and some of its variants have been reported to trigger cytotoxic responses, as well as IE1-354. In the primary immune response to CMV infection in infants, IE1-specific responses are a major component of cellular immune reactivity.

Here, the pattern of immune reconstitution to CMV-pp65 and CMV-IE1 was examined by following the protein-specific ICC and tet binding responses in peripheral blood lymphocytes (PBL) of eleven HLA-A2 subjects for one year following HCT. See Table VI, below. There was no noticeable difference in the distribution of diagnostic disease or hematopoietic cell source between the CMV and No CMV Groups. During the year, 5 subjects had at least one CMV positive blood sample by shell vial and 4 had qualifying QPCR positive assays of blood plasma. In the CMV Group, subject #54 was diagnosed with CMV colitis, and this was the only patient to develop CMV-related disease. His donor as well as the donor from #93 were CMV seronegative.

The responses to the CMV-IE1 peptides were compared to CMV-pp65 peptide in HCT patients with and without CMV reactivation. Even when the multiple IE1-specific peptides were used, the response to IE1 was reduced and delayed compared to the CMV pp65 response.

Eleven CMV seropositive HLA A2 subjects were followed at days 40, 90, 120, 150, 180, and 360 days post HCT. The intracellular cytokine IFN-γ response and results from HLA A2 tetramer binding assays (tet assays) using CMV-derived peptides IE1-256, IE1-297 and IE1-316 were compared to the response to pp65-495 in HCT recipients with and without apparent CMV reactivation. The number of pp65 and IE1 tetramer binding cells were higher in the CMV reactivation group. Neither group produced high levels of CMV-IE1 responsive lymphocytes until late after HCT. The response to the IE1 peptides in the CMV reactivation group reached a median number of reactive cells of 3.8×10⁶ cells/L at day 360 and stayed near the limit of detection in the No CMV group. In the HLA A2 context, therefore, there is a minimal immune response to CMV-IE1 compared to CMV-pp65, in HCT recipients following CMV reactivation.

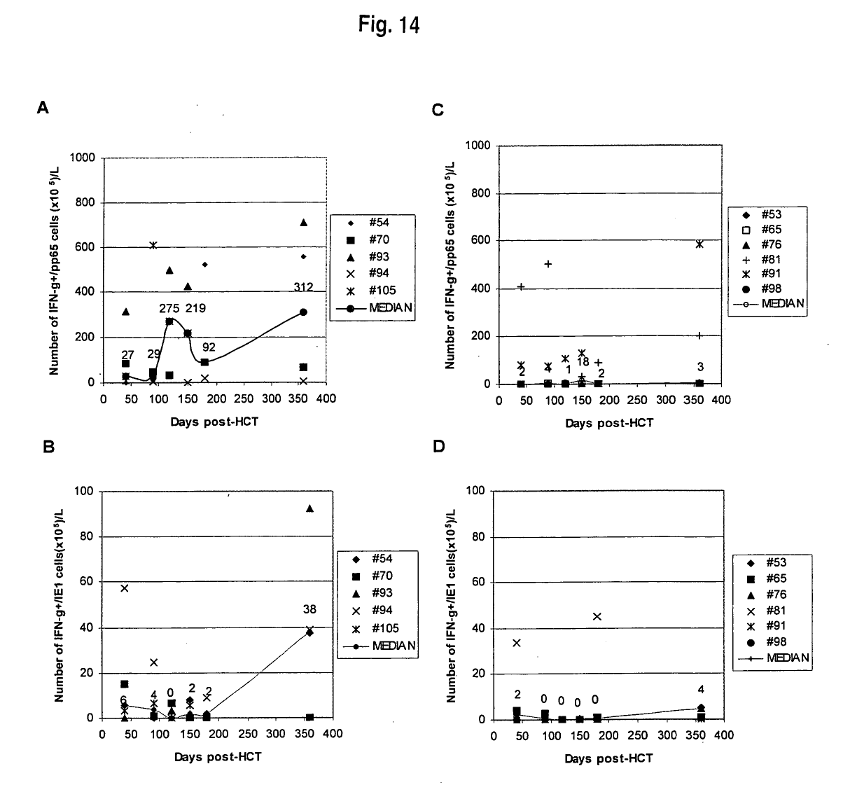

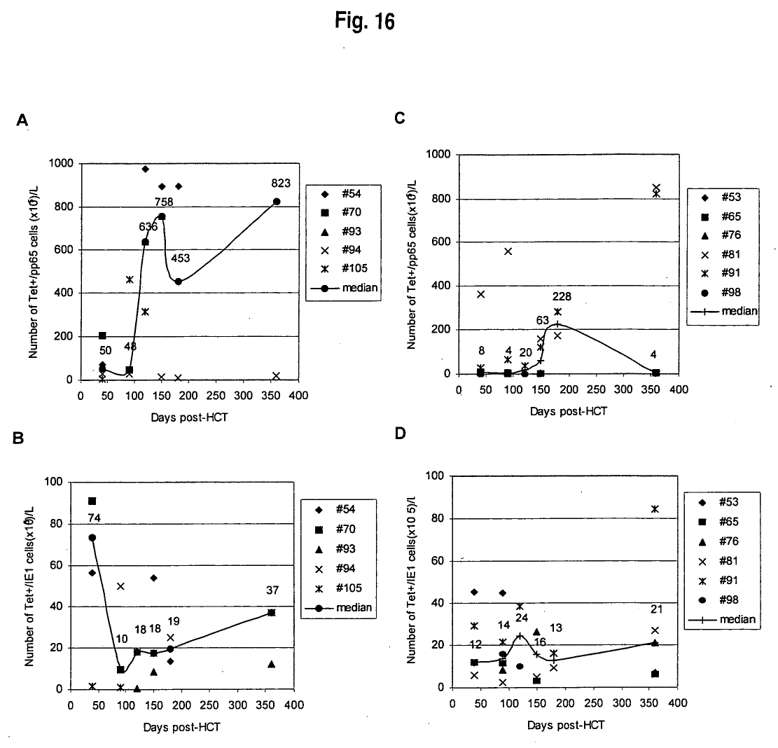

Here, the evaluation of CMV immunity in HCT HLA-A2 subjects relies on two assays, the ICC/IFN-γ and the tetramer binding assays and reactivity to two major CMV proteins, CMV-pp65 and CMV-IE1. The tetramer binding assay has been widely used because it permits the quantitation of CTL simply, by flow cytometry. It is useful in measuring cellular immune response to the CMV pp65 protein because it targets mainly one immunodominant epitope, for example pp65-495 for HLA A*0201 allele. Other epitopes encompassing CMV-pp65 have been described for other HLA alleles suggesting that CMV-pp65 is a major target for immune responses. In contrast, the CMV-IE1 protein presents multiple peptides in the same HLA context and therefore requires multiple tetramer reagents. This places added requirement on the tetramer technology for determining the immune status of an individual. However, the median value of CD8+/tet+ cells binding to tetpp65-495 in the CMV reactivation group, expressed as concentration, was 10-fold higher than the No CMV group and was consistently higher here than the IE1-specific response. When compared to the ICC assay at peak time, only 43% of pp65-495-Te5+ cells were expressing IFN-γ (see FIGS. 16A and 14A). This response rose briskly between days 100-180 post-HCT and remained high at one year.

In the No CMV group, there was a low-level tet+ response to both pp65-495 and to IE1 during this same time, but this never expanded and remained at very low levels at one year. It is likely that exposure to CMV antigen is required for these CD8 expansions, and in the No CMV group, if there was an initial CMV reactivation state, it was then limited, never reached detectible levels in blood and was never sufficient to lead to expansion of CD8-specific cells. The highest levels of tet+pp65-495 cells occur in recipients with CMV reactivation. Without wishing to be bound by theory, it is possible that a previously unrecognized phenomenon of sub-clinical CMV reactivation was shown only by the quantitative variation of CMV reactive T cells.

During CMV reactivation, the CMV-IE1 protein is the first protein to be expressed in infected cells. Therefore, it should be part of the immune response during immune reconstitution. Using the peptides uncovered with the HLA-A2 transgenic mouse model, CMV-IE1-256, 297 and 316 peptides, PBL from HCT recipients were stimulated and tested by ICC/IFN-γ at various times up to one year post-HCT. After CMV reactivation, all three CMV-IE1 peptides stimulated PBL in all 5 subjects at some time between days 40 and 360 post HCT. There was no indication that one peptide was more prominent than the others in the CMV reactivation groups, therefore, a CMV-IE1 peptide mixture preferably should be used to ensure the detection of immune cellular reactivity in all samples. Moreover, in the CMV reactivation group, the immune response to CMV-IE1 was always lower than that to CMV pp65, with the highest media value observed at day 360 (FIG. 14B) suggesting that there may be additional stimulation and expansion after CMV reactivation. The CMV-IE1 ICC cell count was still low at day 180 even though the fact that the median time to the first day of CMV reactivation was day 55 post-HCT.

The reason why there is a reduced response to CMV IE1 compared to pp65 may lie in modification of the response to IE1 by an immune escape mechanism of the virus. If this is the case, the immune system may not be exposed to the IE protein during the reactivation process as it would be during the reactivation process as it would be during a primary infection. In congenital and postnatal CMV infection, IE1-specific responses dominate by one year of age, regardless of the specificity of initial responses. This response to the CMV-IE1 gene is a typical response to primary infection and is in contrast to what is seen in adults with chronic infection. Consistent with this hypothesis, in HCT subjects, although the response to CMV-pp65 always predominates, the response to CMV-IE1 peaks at 1 year post-HCT in the CMV reactivation group. Three subjects in the CMV reactivation group showed ICC positivity to all 3 CMV-IE1 peptides simultaneously at day 360, but not in the No CMV group. These results show a multi-peptidic IE1-specific immune response within the same blood samples. Tetramer binding assays showed that there were CTL cells directed towards IE1-297 and IE1-316 in equal number in both the CMV reactivation and No CMV groups (FIGS. 16B and 16D). So, although the number of CMV-IE1 immune cells are low, they are present in sufficient number to respond to CMV reactivation.

The administration of donor cells, manipulated either by in vitro expansion or by in vivo stimulation with a vaccine, can prevent CMV reactivation. For this to have been effective, the CMV-IE1 proteins are important vaccine constituents since reactivity to these polypeptides is scarce in HCT subjects. The HCT recipient's CMV-reactive cells are of donor origin as shown through PCR vβ repertoire analysis. However, the immune cells specific to CMV pp65 or CMV-IE1 do not amplify similarly in each patient, and thus immunotherapy might require enriched CMV-IE1-specific cells for prevention of CMV disease after HCT. In summary, the immune response to each CMV protein during immune reconstitution after HCT appears to be independent and stimulated by CMV reactivation. Unlike the robust relatively early response to a single epitope of CMV pp65, in HLA A2 recipients the CMV-IE1 response is characterized by a multi-peptide recognition late after CMV reactivation.

Peptides of the invention may be formulated as vaccines according to any suitable method. Naked peptides or lipidated peptides may be formulated with or without a suitable adjuvant or any other pharmaceutical carrier known in the art. A DNA adjuvant is preferred for human use. The peptides may be formulated as fusions with other immunogenic peptides of the invention or with immunogenic peptides from a different pathologic entity. Fusions of peptides with T-helper epitopes such as PADRE or certain known tetanus peptides also are contemplated. Spacer peptides also may comprise part of these fusions.

The peptides may be formulated for any suitable mode of administration, however, subcutaneous, intradermal, mucosal (e.g., rectal, nasal, vaginal, etc.), intraperitoneal, transdermal or inhalant modes of administration are preferred. Those of skill in the art of pharmaceutical formulation are well aware of the appropriate carriers, diluents, excipients and other ingredients which may be used to create formulations for these modes of administration, and any of these compounds and formulations are contemplated for use with the invention.

For human administration, generally a first immunization of about 25 to about 2500 mg peptide is preferred, followed by one, two or more booster immunizations at intervals of about 4 weeks, if desired. Greater or lesser doses are also contemplated, in the range of about 10 to about 10,000 mg per administration.

The peptides of the invention as described above for peptide vaccines also may be administered as a DNA vaccine which encodes the peptide. Such DNA-type vaccines and methods for their formulation are known in the art. Generally, such vaccines are administered to previously infected or uninfected persons, or in vitro to T cells, in the form of a polynucleotide wherein a suitable gene-transfer vector such as a plasmid or engineered virus vector contains DNA that encodes the peptide fragment or fragments under the control of appropriate expression regulatory sequences. T cells transfected in vitro with the DNA-based vaccine may be administered to persons as well.

For DNA immunizations, 6-8 week old mice generally were injected intramuscularly with endotoxin-free DNA diluted in sterile saline, according to known methods, in each thigh. Mice received three separate immunizations at 4 week intervals and the spleens were collected. Subsequent DNA immunizations for individual peptide analysis consisted of one bivucaine HCl (USP 0.05%) MPF injection into the thigh followed 5 days later with one injection of 50 μg pcDNA-IE1 and 50 μg pcDNAGM-CSF. Endresz et al., Vaccine 19:3972-3980, 2001; Thompson et al., Am. J. Physiol. 258:C578-C581,1990. Spleens generally were collected twenty days after the last immunization. Rec-AAV-IE1 DNA was injected only once intramuscularly, as a cleared lysate, at the MOI indicated in the relevant figures with spleen collection thirty days post-immunization.

Two concerns when using a rAAV vector for immunization against CMV are the ability to produce rAAV in amounts necessary for large-scale immunization and the potential long-term effects of integration of rAAV at the injection site, especially if used in young children. By choosing a helper-free encapsidation process similar to those used in clinical gene therapy trials and by demonstrating the ability of rAAV to boost immunity at low input of virus, the first concern is reduced. Titers were based on an infectious assay in which the rAAV-CMV gene expression was detected by immunohistochemistry in a permissive cell line. Since antibodies to both recombinant CMV proteins exist, the infectivity of the rAAV can be assessed directly by the presence of nuclear stain in the infected cell. This method circumvents the possible pseudo-transduction of a cytoplasmic marker protein such as β-galactosidase or hrGFP that may interfere with the vector titration.

For primary immunization alone, only high titers (1.5×10⁸ IU/mouse) gave rise to CTL responses as has been reported previously with CsCl purified virus. The low dose immunization required initial priming of the immune response with a DNA vaccine. This DNA priming is similar to a CMV latent infection in human subjects. In this human setting, boosting the immune system of seropositive donors towards CMV infection with low dose rAAV-CMV (for example three to five log lower) would greatly benefit hematopoietic cell transplantation subjects in whom CMV is still a life-threatening disease.

With respect to potential risks of integration of rAAV in the vaccine, there is evidence that integration into muscle is rare. Injection of as much as 10¹¹ rAAV into muscle in hemophiliacs has not been associated with severe toxicity. Lower doses of vaccine also therefore are likely to be safe. In the mouse model, there is no observed muscular dysfunction. rAAV for immunization in humans therefore is feasible for subjects at risk for CMV disease.

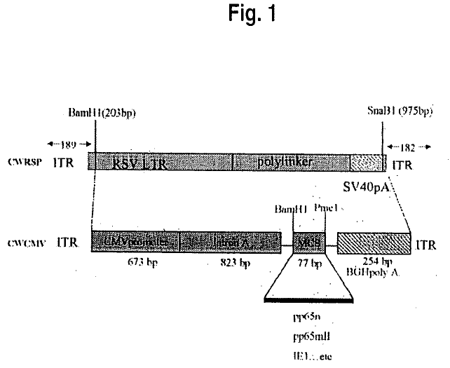

The encapsidation of the rAAV for vector generation involves (1) the transfection of a plasmid containing the gene of interest flanked by the rAAV vector ITR into HEK-293 cells that express adenovirus E1a; (2) simultaneous transfection of a plasmid that contains the AAV rep and cap genes; and (3) a viral infection with adenovirus, herpes virus or a plasmid that provides helper viral functions such as adenovirus E1, E2, E4 and VA RNAs. See FIG. 1 for a map of a recombinant plasmid according to an embodiment of the invention.

For rAAV vaccine generation, the plasmid-based method can be performed using a commercially available helper virus-free, three plasmid transfection kit. rAAV was generated to encode two CMV genes, the immediate-early 1 (CMV-IE1) and the kinase-deficient pp65mII (CMV-pp65mII). A two-dose regimen may not be sufficient to produce a significant CTL response. Therefore, to improve the immune response while reducing the number of injections required, in one embodiment, the A2/Kb and HHDDII mice are immunized with a single dose of semi-purified rAAV-IE1 (1.5×10⁸ IU/mouse). This resulted in significant CTL responses in splenocytes 30 days later. This shows that low input rAAV-CMV-pp65 and -IE1 with a prime-boost strategy can induce cellular and humoral immunity to these CMV proteins.

Cellular vaccines and antigen presenting cells incorporating the inventive peptides also form part of the invention. Such cells and cellular vaccines are antigen-presenting cells that have been treated in vitro to cause them to present the inventive peptides according to known methods in the art, for example, by in vitro incubation with (50 μM) peptide or peptides for about 1-2 hours, followed by washing. Alternatively, the cells may be infected with a transfer virus vector containing DNA that encodes the peptide(s). The DNA construct for DNA vaccines may consist of a mammalian expression vector such as PVAX (InVitrogen™) in which the DNA sequence of each of the peptides of interest are inserted in the multicloning site, separated by spacers. For production of cellular vaccines, the described DNA construct may be electroporated into appropriate cells such as autologous dendritic cells.

An additional aspect of the invention relates to diagnostic reagents for detection of CMV infections. The peptides according to the present invention can stimulate CTL directly in vitro and therefore can be used in an assay to determine the degree of immunostimulation being caused by HCMV. The peptides also can be used to diagnose individuals who are infected with CMV. For use as a diagnostic reagent, for example for the detection of active versus quiescent CMV infections, the peptides may be contacted in vitro with a patient sample containing T cells, or antigen-presenting cells presenting peptides of the invention may be contacted in vitro with such a sample. Expansion of T cell clones recognizing the peptide from the patient sample indicates the presence of CMV-reactive CTL and therefore CMV infection. For example, Bissinger et al., Exp. Hematol. 30:1178-1184, 2002, the disclosures of which are hereby incorporated by reference, have described the use of an intra-cellular cytokine assay to expand HCMV-specific CTL with IL-2 and feeder cell stimulation using pp65 specific peptides. Using this method, not only can the ICC assay determine whether the subject is reactive to HCMV, cells also can be isolated and expanded to be used for adoptive immunotherapy. Alternatively, tetramer reagents, dimer reagents and the like, which are known in the art, for example, those disclosed in U.S. Pat. No. 5,734,023, the disclosures of which are hereby incorporated by reference, may be constructed from the peptides of the invention to enable detection of CMV-specific T cells. Class I tetramer folded in the presence of CMV pp65 peptide can detect CTL specific to CMV infection. See Lacey et al. Transplantation 74:722-732, 2002.

Tetramer-positive cells also may be transferred into the recipient into which expansion is desired. The presence of CTL does not prevent HCMV reactivation, but there is evidence that they protect against HCMV disease.

### EXAMPLES

### Example 1

### Construction of Recombinant Adeno-Associated Virus Expressing CMV IE1

Recombinant adeno-associated virus construct (recAAV-IE1) was constructed as follows. An internal cassette containing RSVLTR, a polylinker and SV40pA was removed from the recAAV CWRSP plasmid backbone with BamH1/SnaB1, leaving the ITR from AAV2 intact, and replaced by the CMV promoter, intronA, MCS and BGHpA cassette from pcDNA 3.1+ as described by Chatterjee et al., Science 258:1485-1488, 1992. The IE1 gene then was placed in the MCS at the EcoRI/XbaI site (CwCMV-IE1) and its expression was verified by transfection of HEK293 cells using a Cellphect™ transfection kit.

HEK293 cells containing the adenovirus E1A gene were transfected with 10 μg of CWCMV-IE1, 10 μg of pHelper™ (containing E2A, E4 and VA RNA from adenovirus) and 10 μg of PAAV-RC containing the rep/cap genes from AAV2 for 72 hours to encapsidate the AAV virus using the AAV Helper-Free System (Stratagene7, Cedar Creek, Tex.). The cells then were collected, resuspended in 0.1 M Tris-HCl pH 8.0, frozen/thawed four times and sonicated for 30 seconds. The lysate was cleared by centrifugation at 7000 g for 20 minutes at room temperature. The supernatant was aliquoted and stored at −80° C. HT1080 cells, made permissive according to the Stratagene™ protocol (using RPMI-1640 supplemented with 40 mM hydroxyurea and 1 mM sodium butyrate), were stained for the IE1 gene product after 48 hours using commercial anti-CMV early nuclear protein monoclonal antibodies and visualized with a commercial peroxidase kit to determine the recAAV-IE1 titer in the cells (1.5×10⁹ IU/ml).

### Example 2

### Stabilization of HLA-A2 Expression by IE1-Derived Peptides

T2 cells are defective for endogenous class I presentation but the presence of peptide binding to the MHC molecule will stabilize its expression on the cell surface. The stabilized MHC molecule can be detected by flow cytometry using a monoclonal antibody to the HLA A-A*0201 molecule. Peptide sequences were selected using two algorithms for HLA peptide predicted motifs publicly available on the internet (SYFPEITHI (Rammensee et al., Immunogenetics 50:213-219, 1999) and BIMAS). The first 5 peptides with the highest scores common to both databases were synthesized (IE1-81, IE1-256, IE1-297, IE1-304 and IE1-316). See Table II.

TABLE II
Peptide Sequences.

| | | | |
|---|---|---|---|
| Peptide | Peptide Name | Sequence | SEQ ID NO: |

| | | | |
|---|---|---|---|
| IE1ₚ₈₁₋₈₉ | IE1-81 | VLAELVKQI | 4 |
| IE1ₚ₂₅₆₋₂₆₄ | IE1-256 | ILDEERDKV | 1 |
| IE1ₚ₂₉₇₋₃₀₄ | IE1-297 | TMYGGISLL | 2 |
| IE1ₚ₃₀₃₋₃₁₁ | IE1-303 | SLLSEFCRV | 5 |
| IE1ₚ₃₀₄₋₃₁₂ | IE1-304 | LLSEFCRV | 6 |
| IE1ₚ₃₁₅₋₃₂₃ | IE1-315 | YVLEETSVM | 7 |
| IE1ₚ₃₁₆₋₃₂₄ | IE1-316 | VLEETSVML | 3 |
| IE1ₚ₃₅₄₋₃₆₃ | IE1-354 | YILGADPLRV | 8 |
| IE1ₚ₃₅₅₋₃₆₃ | IE1-355 | ILGADPLRV | 9 |
| CMVpp65₄₉₅₋₅₀₃ | CMVA2-495 | NLVPMVATV | 10 |
| CMVpp65₂₆₅₋₂₇₅ | CMVB7-265 | RPHERNGFTVL | 11 |
| CMVpp65₄₁₇₋₄₂₆ | CMVB7-417 | TPRVTGGGAM | 12 |
| HIVpol₄₆₈₋₄₇₆ | HIV468 | ILKEPVHGV | 13 |

Four IE1-derived peptides (IE1-81, IE1-256, IE1-297, and IE1-304) were tested individually for binding and stabilizing effect of the MHC molecule on T2 cells. The peptides were assayed on T2 cells for their ability to bind and stabilize the A2 molecules on the cell surface according to methods described in Gricks et al., Cancer Res. 61:5145-5152, 2001, with modifications. Cells (2×10⁵) were incubated for 18 hours in 100 μL RPMI 1640 with 1% FBS in a 96-well plate with 100 μM of each peptide at 37° C. in 5% CO₂. The level of stabilized HLA-A2 on the surface of the T2 cells was determined according to known methods using monoclonal antibody BB7.2, which specifically recognizes HLA A*0201 as described in BenMohamed et al., Hum. Immunol. 61:764-779, 2000 and Parham et al., Hum. Immunol. 3:277-299, 1981 and a FITC-labeled goat anti-mouse F(ab′)₂. Fluorescence was detected with a FACSCalibur™ flowcytometer. Numbers in the peak channel were compared to control T2 cells that contained HLA mismatch B7 peptide or no peptide.

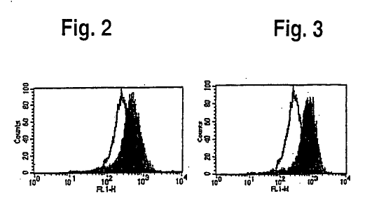

FIG. 2 shows the displacement of the peak fluorescence to the right in T2 cells incubated with IE1-81 (black area) compared to the background signal on T2 cells treated with a mismatched HLA (B7-restricted) peptide (white area). FIG. 3 shows the same information for IE1-297. Data are not shown in graphical form for peptides IE1-256 and IE1-304. The results were expressed as fold increased fluorescence compared to the mean of no peptide and HLA mismatch B7 peptide.

TABLE III
Peptide Peak Channel Values.

| | peak | Fold |
|---|---|---|
| | channel | Increase |
| IE1-81 | 495 | 2 |
| IE1-256 | 784 | 3.3 |
| IE1-297 | 813 | 3.4 |
| IE1-304 | 433 | 1.8 |
| (+)control pp65 | 1512 | 6.4 |
| (−)control B7 T10M | 232 | 1 |
| (−)control B7 R11L | 261 | 1 |
| (−)control no | 237 | 1 |
| peptide | | |

All four IE1-derived peptides stabilized the HLA-A2 molecule (with varied binding affinity). See Table III. The peak values of IE1-297 (×3.4) and IE1-256 (×3.3) were highest but lower than the positive control pp65₄₉₅₋₅₀₃ (×6.4). Therefore, all four peptides (IE1-81, IE1-256, IE1-297, IE1-304) and later IE1-316 as well were used together, each at a concentration of 25 μM (“IE1 mix”) to bind to autologous blasts cells for in vitro stimulation. The IE1 mix also was used to sensitize target T2 cells for cytotoxicity recognition.

TABLE IV
DNA Immunizations.

| Mouse | DNA |
|---|---|
| M1 | 100 μg pcDNA-IE1 + 100 μg pcDNAGM-CSF |
| M2 | 100 μg pcDNA-IE1 + 100 μg pcDNAGM-CSF |
| M3 | 100 μg pcDNA-IE1 + 100 μg pcDNAGM-CSF |
| M4 | 100 μg pcDNA-IE1 + 100 μg pcDNAGM-CSF |
| M5 | 100 μg pcDNA-IE1 + 100 μg pcDNApp65mII |
| M6 | 100 μg pcDNA-IE1 + 100 μg pcDNApp65mII |
| M7 | 100 μg pcDNA-IE1 + 100 μg pcDNApp65mII |
| M8 | 100 μg pcDNA-IE1 + 100 μg pcDNApp65mII |
| M9 | 50 μg pcDNA-IE1 + 50 μg pcDNApp65mII + 50 μg |
| | pcDNAGM-CSF |
| M10 | 50 μg pcDNA-IE1 + 50 μg pcDNApp65mII + 50 μg |
| | pcDNAGM-CSF |
| M11 | 50 μg A-IE1 + 50 μg pcDNApp65mII + 50 μg |
| | pcDNAGM-CSF |
| M12 | 50 μg pcDNA-IE1 + 50 μg pcDNApp65mII + 50 μg |
| | pcDNAGM-CSF |

### Example 3

### DNA Immunization with IE1, pp65m II and GM-CSF Combinations

Genes encoding pp65mII, IE1 and murine GM-CSF were inserted into the mammalian expression vector pcDNA3.1+ as described previously in Gallez-Hawkins et al., Scand. J. Immunol. 55:592-598, 2002. Pp65MII, a kinase-deficient mutant pp65 protein, was introduced into pcDNA at the Nhe1/EcoR1 site as a whole cassette containing intronA/pp65mII. PcDNA3.1+ was modified as follows for the other constructs. IntronA of the immediate-early gene was inserted by PCR at the Nhe1/BamH1 site of the pcDNA3.1+ MCS. The IE1 gene was removed from vector pNEB-IE1 at the Pme1/Sma1site and inserted into pcDNAintA at the EcoRV site as a double blunt end ligation. Murine GM-CSF cDNA was amplified with primers containing the specific RE sites Not1 and Apal (5′ TATAGCGGCCGCCTCAGAGAGAAAGGCTAAGGT; SEQ ID NO:14 and 3′ TATAGGGCCCTATCTCTCGTTTGTCTTCCG; SEQ ID NO:15). All plasmids were transformed in DH5α competent cells and grown in appropriate LB media. The DNA was isolated using Qiagen™ endo-free Maxi™ kit and was tested for expression on A293 human embryonic kidney cells using DMRIE-C (Gibco-BRL™) as a transfection agent. The cells were stained with monoclonal antibody 28-103 to detect pp65 and with anti-CMV early nuclear protein monoclonal antibody to detect IE1 protein. The bound antibodies were visualized with a commercial peroxidase kit. GM-CSF protein was detected in the supernatant of transfected A293 cells by ELISA using Pharmingen™ antibodies.

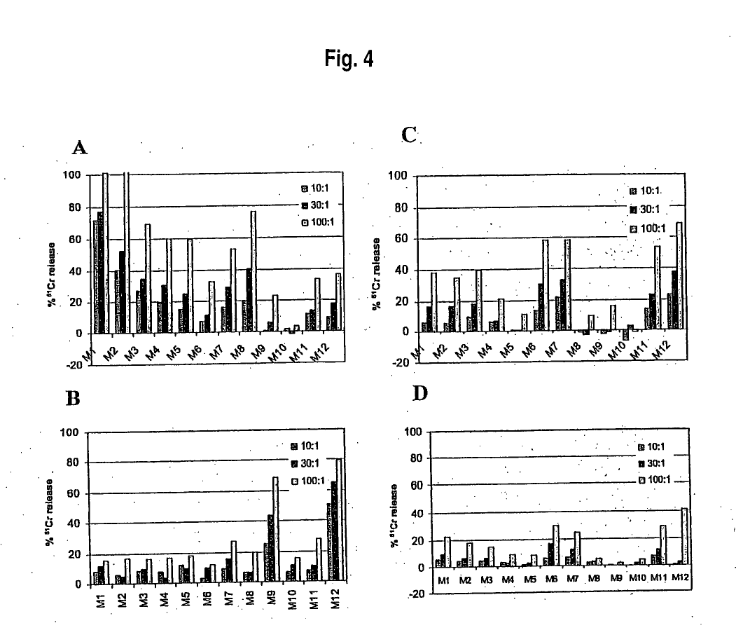

Six- to 8-week old A2/Kb mice were immunized 3 times intramuscularly at 4 week intervals with various combinations of DNA expressing CMVpp65mII, CMV-IE1 and GM-CSF as indicated below. FIG. 4 shows the results of CTL killing mediated by splenocytes collected 10 days after the last (fourth) immunization (FIGS. 4A and 4B) to measure short-term responses or collected 60 days the last (third) immunization (FIGS. 4C and 4D) to measure long-term memory response. Chromium release assays were carried out essentially as described previously, in a four hour incubation with ⁵¹Cr-labeled target cells. See Gallez-Hawkins et al., Scand. J. Immunol. 55:592-598, 2002. In the assay, blast feeder cells and target A293-IE1 cells were incubated with a mixture of IE1 peptides at a concentration of 25 μM each. Control A293-IE1 cells were incubated with ⁵¹Cr only. Individual peptide experiments were performed using T2 targets cells incubated with 100 μM of the designated peptide.

The specific targets used in this experiment were T2 cells incubated with IE1 mix (see FIGS. 4A and 4C) or pp65₄₉₅₋₅₀₃ (FIG. 4B), or A293 cells constitutively expressing the IE1 gene (FIG. 4D). Control target T2 cells (no peptides) or non-transfected A393 cells were not lysed by the effector cells (data not shown). pp65mII DNA was a positive control that resulted in immune response in 50% of immunized mice. The % chromium release is reported at an effector/target ratio of 10:1, 30:1, 100:1 as indicated in each Figure.

Specific results were as follows. For FIG. 4A, immunized A2/Kb mice were boosted 10 days prior to splenocyte collection. The splenocytes were incubated with IE1 mix and blasts during a 6 day in vitro stimulation and then subjected to a chromium release assay in the presence of T2 target cells labeled with IE1-mix peptides. Eleven out of 12 mice showed various levels of CTL activity to the mixture of IE1 peptides ranging from 20% to 100% ⁵¹Cr release, demonstrating a strong CTL immune response to the CMV-IE1 immunization. FIG. 4B represents the same spleen cells stimulated with the peptide specific to CMV-pp65mII(P₄₉₅₋₅₀₃) instead of IE1 mix. Only the mice immunized with the pp65mII DNA (M5 to M12) showed CTL activity toward T2-P₄₉₅ targets, demonstrating specificity of response with the respective peptides. Cytotoxicity less than 20% was considered a negative response to the immunization regimen.

In FIG. 4C, immunized splenocytes collected at day 60 after the last injection were stimulated with IE1 mix as described above and incubated in the presence of target T2-IE1 mix cells. Memory T cells directed to the CMV-IE1 gene was detected in these cells. FIG. 4D shows that memory effector cells also recognized endogenously processed IE1 in human A293 targets (A293-IE1). This suggests that the pool of IE1 peptides includes peptides that are naturally processed through the proteosome of the cell.

These data indicate, therefore, that stimulation with the IE1 mixture of peptides generates CTL that can recognize T2 cells labeled with the IE1 mix, that the CTL also can recognized endogenously processed IE1 and that this effect is specific to CMV-IE1 DNA immunization.

### Example 4

### Immunodominant IE1 Epitopes in A2/Kb Mice

Splenocytes from A2/Kb mice were immunized once with 50 μg of pcDNA-IE1 and GM-CSF and used to identify the individual peptide(s) responsible for CTL recognition. At day 20 after immunization, spleen cells were collected and stimulated for 6 days with an IE1 mix and blasts. The splenocytes from responsive mice, as determined by lysis of IE1 mix-loaded T2 cells, were tested with T2 cells loaded with individual peptides. Mouse 1 (Ml) spleen cells were stimulated with the pool of IE1 peptides 6 times whereas the other splenocytes were stimulated only once.

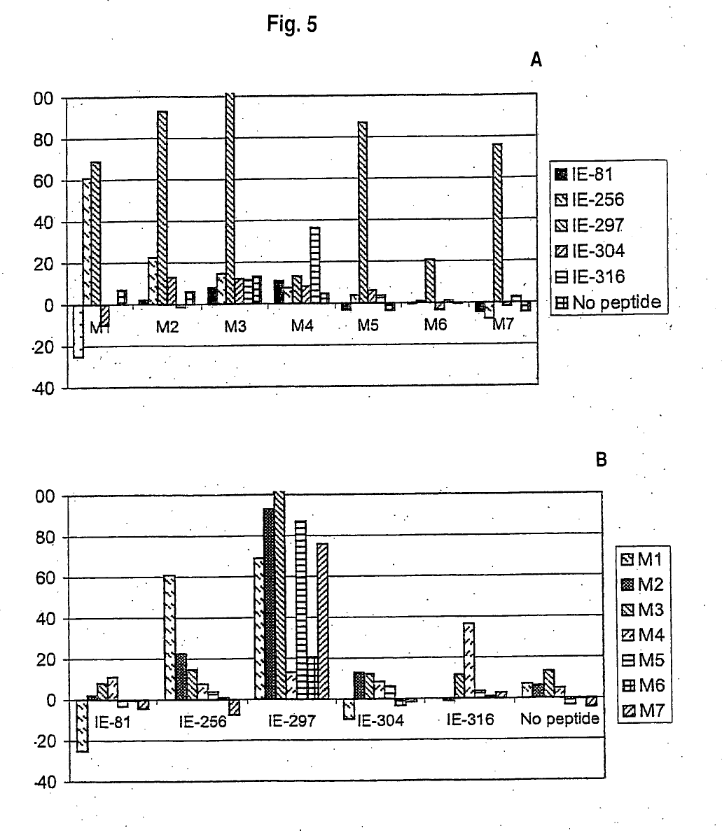

The results of target cell lysis by splenocytes from seven different IE1 immunized mice are shown in FIG. 5A. The specificity of the stimulated population was determined by Cr-release using T2 cells sensitized with each individual peptide as targets (E:T of 100). Mouse Ml recognized T2 targets loaded with IE1-256 or IE1-297. M2, M3, M5, M6 and M7 splenocytes recognized IE1-297 and M4 recognized IE1-316. FIG. 5B shows that the peptide most frequently recognized by A2/Kb spleen cells was IE1-297, though IE1-256 and IE1-316 were also present less frequently. In this group of effector cells, no response to IE1-81 or IE1-304 were detected.

### Example 5

### IE1 Epitope Recognition in A2/Kb Transgenic Mice After recAAV Vector Immunization

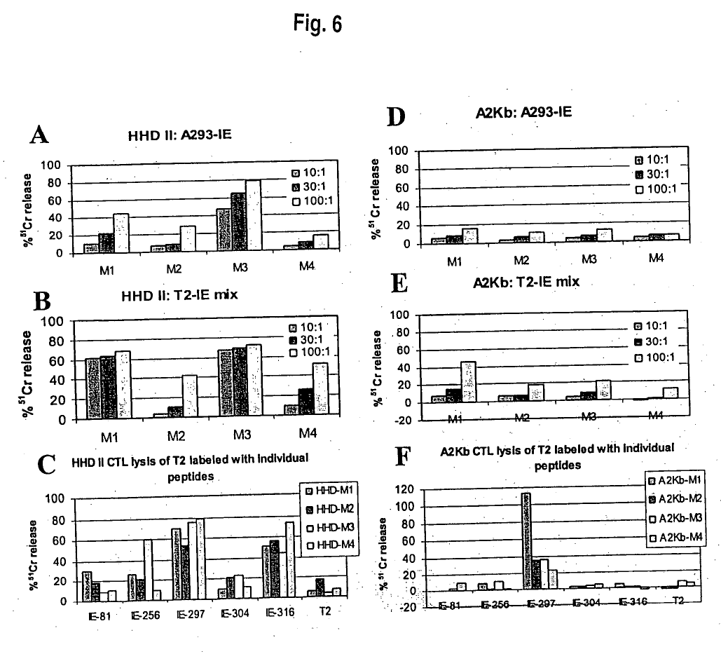

The immune response to full-length IE1 induced by recombinant adeno-associated virus (recAAV-IE1) was characterized for preferential peptide presentation in HHD II and A2/Kb mice. The HHD II mice (HLA-A-0201 α1-α2, H-2D^{b} α3-transmembrane and intracytoplasmic domains) in which the H-2D^{b} and mouse β2m genes have been disrupted by homologous recombination and an internal cassette removed from the CWRSP plasmid backbone, leaving the internal transcription region from AAV2 intact, and replaced by the CMV promoter, intronA, multiple cloning site, and BGHpA cassette from pcDNA3.1⁺ were used. The IE1 gene was placed in the multiple cloning sites at the EcoRI/XbaI site(CwCMV-IE1). An AAV helper-free system (Stratagene™) was used to encapsidate the AAV. The viral lysate was cleared by centrifugation, and the titer of the supernatant was determined on HT1080 cells. The viral vector recAAV containing the IE1 gene was used as another mode of immunization to check for preferential peptide presentation. Four HHD II and four A2/Kb mice were immunized intramuscularly with a single dose of 1.5×10⁸ IU recAAV-IE1 per mouse. The spleen cells were collected 30 days after immunization, stimulated with autologous blast cells loaded with the IE1 mix peptides for 6 days and assayed for IE1 CTL response. FIGS. 6A-6C show the results for HHD II mice and FIGS. 6D-6F show results for the A2/Kb mice. After one IE1 mix stimulation, three out of four HHD II mice generated CTLs recognizing endogenously processed IE1 peptides (A293-IE; see FIG. 6A) whereas four out of four HHD II mice responded to the IE1 immunization with IE1 mix T2 target cells (see FIG. 6B). When the same splenocytes were incubated with T2 cells labeled with individual peptides, IE1-297 and IE1-316 were the most frequently recognized peptides (four mice), followed by IE1-256 (one mouse). See FIG. 6C.

For the A2/Kb mice, the response to A293-IE1 target cells was low (FIG. 6D), however one out of four mice showed a substantial CTL recognition with IE1 mix-T2 targets after IE1 mix stimulation (FIG. 6E). The CTL response was specific for the IE1-297 peptide in all 4 A2/Kb mice (FIG. 6F). These results show the preferential recognition of peptide IE1-297 in CTL generated by CMV-IE1 immunization.

### Example 6

### Intracellular Cytokine Response to IE1-297 in Human HLA-A*0201 PBL

Having identified IE1-297 peptide as an important epitope in the process of CTL response to CMV-IE1 immunization in A2 transgenic mice, this peptide was tested to determine if it was recognized by CD8 cells from CMV seropositive human subjects. Fresh whole blood samples were collected from four individuals susceptible to CMV reactivation 40, 120, 150 and 180 days after stem cell transplantation. Frozen white blood cells from the human subjects were stimulated for 6 hours with 100 μM IE1-297, pp65(p₄₉₅) or HIV peptide (negative control) and PHA (positive control). The cells then were stained to detect CD8 and IFN-γ production. Four patient samples were analyzed by cytokine flow cytometry.

Intracellular cytokine (ICC) assays were performed essentially as described in Dunn et al., J. Infect. Dis. 186:15-22, 2002, using 200 μL of fresh blood stimulated with 100 μM of pp65(_{P495-503}) or IE1(_{P297-305}) peptides for two hours. Brefeldin A was added and the blood cells were incubated for another four hours. No costimulatory antibodies were used. Once stimulated in this way, the blood lymphocytes were stained for CD8, fixed and then permeabilized using FACS™ lysing solution (BD Biosciences™). The fixed cells were stained for intracellular IFN-γ with an anti-IFN-γ-APC antibody conjugate and analyzed on a FACSCalibur™ flowcytometer. Background was deducted from the calculated % positive cells.

Two out of four samples showed a cytokine response to the IE1-297 peptide whereas 4 out of 4 responded to the pp65₄₉₅₋₅₀₃ peptide. See Table V. In subject 93, 4.77×10⁷ cells per liter were IFN-γ responsive to pp65 and 2.11×10⁶ cells per liter were responsive to IE1-297. These data indicate that HLA A*0201 PBL can respond to IE1-297 stimulation and that this peptide can be used to characterize the status of a CMV cellular response in human subjects.

TABLE V
ICC using Fresh Blood from CMV Seropositive Stem
Cell Transplantation Subjects.

| | | pp65(p495) | | IE1(IE1-297) | | |
|---|---|---|---|---|---|---|
| Patient | Days post- | % | Total number | % | Total number | PHA^{c} %, CD8⁺/ |
| No. | transplant | CD8⁺/IFN-γ | of cells/L^{a} | CD8⁺/IFN-γ | of cells/L | IFN-γ |
| 100 | 40 | 0.11 | 4.08 × 10⁵ | 0.00 | 0.00 | 2.09 |
| 93 | 120 | 3.62 | 4.77 × 10⁷ | 0.16 | 2.11 × 10⁶ | 37.6 |
| 70 | 150 | 0.47 | NA^{b} | 0.20 | NA^{b} | <33.15 |
| 70 | 180 | 1.26 | 6.97 × 10⁶ | 0.01 | 5.53 × 10⁴ | 19.2 |

^{a}The total number of IFN-γ+ cells was calculated taking into account the percent of total lymphocytes in the blood and the percent CD8⁺cells from that fraction and expressed as number of cells per liter of blood. A total of 50,000 events were counted for each samples.
^{b}Total WBC count and % lymphocyte count not available.
^{c}PHA, phytohemagglutinin.

### Example 7

### Prime-Boost Immunization using pp65mII DNA Followed by rAAV-pp65II

The CMV gene for pp65mII was cloned as previously described by Yao et al., Vaccine 19(13-14):1628-1635, 2001, and inserted in pcDNA3,1+ (Invitrogen™). The pcDNA3.1+ was modified to contain the CMV intronA downstream from the promoter to stabilize the expression of the above CMV genes. The DNA plasmids were grown in LB broth and the purified DNA using an endotoxin-free Qiagen™ kit. The DNA was resuspended in sterile saline for immunization purposes.

enhance CMV gene expression in AAV2 plasmid, the expression cassette was modified as follows using the rAAV CWRSP plasmid. The internal cassette containing the RSV LTR, polylinker and SV40pA was removed from the plasmid backbone with BamH1/SnaB1, leaving the ITR from AAV2 intact and replaced with the CMV promoter, intronA, MCS and BGHpA cassette from pcDNA3.1+ as shown in FIG. 1. The CMV-IE1 or CMV-pp65mII gene was placed in the MCS at the EcoR1/Sba1 site (CWCMV-IE or CWCMV-pp65mII) and their expression was verified by transfection of HEK-293 cells using a commercial kit. To encapsidate AAV, the AAV helper-free system (Stratagene™) was used to transfect HEK-293 cells (containing the adenovirus E1A gene) with 10 μg of pHelper™ (containing E2A, E4 and VA RNA from adenovirus) and 10 μg of pAAV-RC containing the rep/cap genes from AAV2. The cells were collected after 72 hours, resuspended in 0.1 Tris-HCl (pH 8.0), subjected to a freeze-thaw cycle four times, sonicated for 30 seconds and then clarified by centrifugation twice at 10,000×g for 30 minutes at room temperature. The semi-purified viral stock was aliquoted and stored at −80° C. The titer was determined using HT1080 cells, made permissive in the presence of 240 mM hydroxyurea and 6 mM sodium butyrate, then stained for the expression of CMV genes after 48 hours using appropriate monoclonal anti-CMV protein antibody and revealed with a commercial peroxidase kit.

The encapsidation of the AAV recombinant DNA was performed in the absence of helper virus with a three-plasmid transfection kit. The rAAV inoculum was evaluated for the expression of the CMV-IE1 and CMV-pp65mII gene and the titer was determined using expression of the transgene. Viral inoculum of reporter proteins such as β-galactosidase and alkaline phosphatase inserted in rAAV can be present as free proteins in the inoculum.

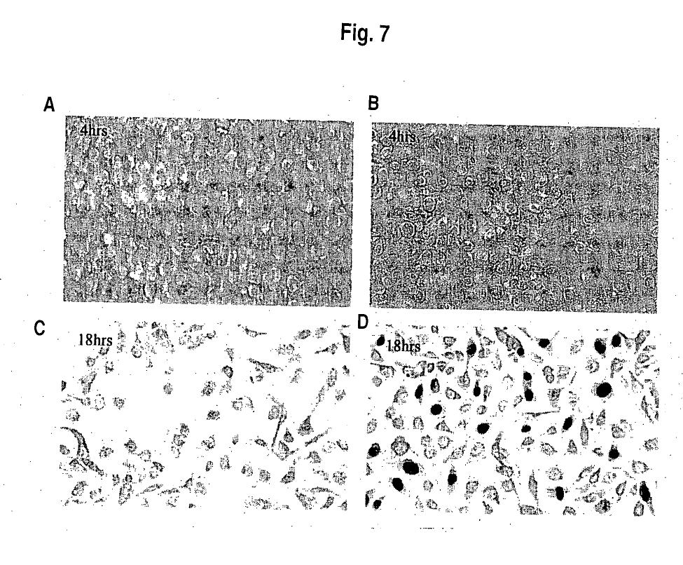

HT1080 permissive cells were transduced with rAAV-IE (7.5×10⁴ IU/well in a 12-well plate) in the presence and absence of 10 μM cycloheximide (CH), a translation inhibitor of protein synthesis. The cells were stained for IE1 expression 4 and 18 hours post-translation. FIG. 7 (7A and 7C: 10 μM cycloheximide; 7B and 7D: 0 μM cycloheximide) shows that rAAV-IE was only expressed at 18 hours post-transduction and in the absence of CH. No CMV-IE1 nuclear protein was found in the CH-treated cells, demonstrating that the expression of the CMV protein was the result of rAAV-IE de novo expression. The same results were obtained with rAAV-pp65mII.

The rAAV viral inocu-lum also was tested for the presence of non-encapsicated plasmid DNA that may have contaminated the viral preparation and therefore affected the titer assay. The viral preparation was treated with DNase I for 30 minutes at 37° C. according to known standard protocols. The rAAV titer in the DNase-treated sample was similar to the untreated sample suggesting that the titers of rAAV were correctly assessed in the semi-purified sample. The titer of rAAV-IE1 was 1.5×10⁹ IU/mL. For the CMV-pp65mII gene inserted into the CWCMV plasmid, the encapsidated vector had a titer of 5×10⁶ IU/mL. A control AAV2 expressing the LacZ protein had a titer of 2.5×10⁷ IU/mL. All vectors were used at the same input for animal immunizations.

The prime-boost immunization schedule was tested in transgenic A2/Kb mice using 100 μg pcDNAintAppmII DNA with 100 μg pcDNAingAgm-CSF DNA per mouse for priming followed by rAAV boost. Kinase-deficient pp65 DNA has been shown previously to trigger CTL activity in chromium release assays using target cells presenting the CMV-pp65-495 peptide. The chromium release assays were carried out according to known methods as follows. The effector cells were collected at day 6 after the first in vitro stimulation that consists of irradiated autologous blast cells (stimulated for 3 days with 25 μg/mL LPS and 7 μg/mL dextran sulfate) in the presence of 100 μM peptide pool. The target cells were human T2 cells expressing HLA A2, loaded with CMV derived peptides specific to the CMV gene used for immunization. The negative control was T2 cells without peptides or with peptides derived from CMV proteins not used in the immunization. The T2 cells were incubated with peptides and 100 μCi ⁵¹Cr for 1 hour, then washed for 30 minutes in RPMI-2% FBS before they were co-cultured with effector cells at a ratio of effector/target of 100:1, 30:1 and 3:1 for 4 hours at 37° C. and 5% CO₂. The supernatant (25 μL) was added to 100 μL of scintillation fluid and counted in a BetaTOP™ counter. The remaining effector cells were stimulated in vitro a second time for 5 days and tested again for chromium release.

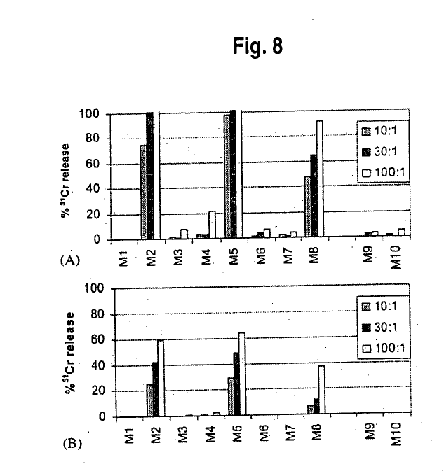

To test the ability of the rAAV to stimulate memory T cells, the mice were boosted with a low dose of rAAV-pp65mII (3×10⁵ IU/mouse). A2/Kb mice were injected with 100 μg pcDNAintgm-CSF, followed 84 days later by rAAV-pp65mII inoculation (3×10⁵ IU/mouse) in the thigh. Mice M9 and M10 received the control vectors without CMV genes. The spleens were collected 20 days later for CTL assays. Three out of eight mice had a significant CTL response reaching 100% lysis of T2 target cells labeled with CMV-pp65-495 (see FIG. 8A) and with human LCLA2 targets (EBV-transformed HLA A*0201 B cells) presenting the same peptide (see FIG. 8B). The amount of rAAV used here was three logs lower than the amount used in single injection immunizations, demonstrating that it is possible to boost memory T cells with a low dose of rAAV. The control mice M9 and M10 (see FIG. 8) received pcDNAintA and pcDNAintAgm-CSF control DNA followed by a control rAAV-lacZ boost. These results show that rAAV-pp65mII, generated with a helper-free system and low virus input, can induce a significant immune CTL response in transgenic HLA*A0201 mice with a prime-boost scheme.

### Example 8

### Prime-Boost Immunization with Simultaneous Injection of CMV-IE1 and CMV-pp65mII Genes

To determine whether both rAAV vectors could be used together, mice were tested for simultaneous CTL responses to both the CMV-IE1 and the pp65mII genes using this same method. Eight transgenic A2/Kb mice (6-8 weeks of age) were inoculated in each thigh with 50 μg pcDNAintA-IE1, 50 μg pcDNAintA-pp65mII, and 50 μg pcDNAintAgm-CSF DNAs in 100 μL saline. Two mice received the control DNAs, pcDNAintA, and pcDNAintAgm-CSF. Thirty days later, they received a booster injection of either 3×10⁵ IU rAAV-IE and 3×10⁵ IU rAAV-pp65mII (test mice) or 3×10⁵ rAAV-lacZ (control mice M9 and M10). Animals were sacrificed 19 days later and splenic lymphocytes were evaluated for specific immune response.

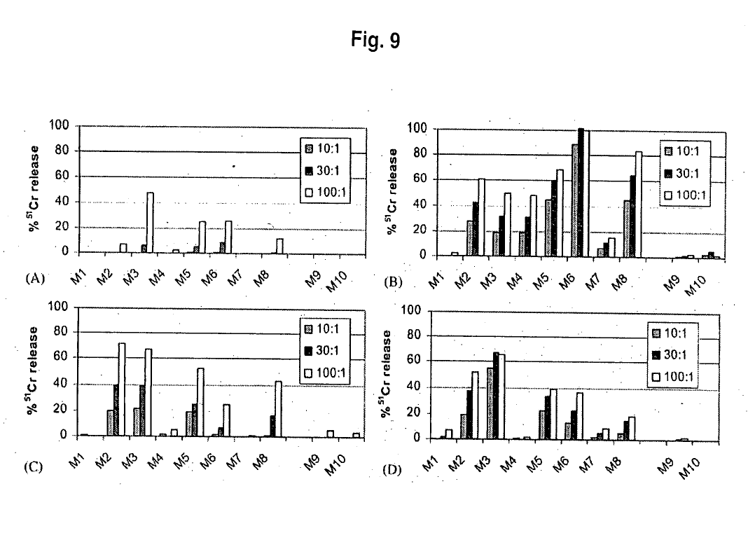

The CTL response to the respective peptides after one in vitro stimulation are shown in FIGS. 9A and 9B, and the response after a second in vitro stimulation in FIGS. 9C and 9D. The splenocytes were stimulated with IE1-mix and lysed T2 cells loaded with CM-IE1-mix (FIGS. 9A and 9C) or stimulated with CMVA2-495 and lysed targets loaded with the same peptide (FIGS. 9B and 9D). The CTL lysis using target cells T2-IE mix was higher in after a second in vitro stimulation (FIG. 9C) in 6 out of 8 mice, whereas the CMV-pp65mII response was about the same after each stimulation in 5 of 8 mice (FIG. 9D). Mice Ml and M7 did not respond to either gene and M4 responded to the IE1 vaccine only, results that are typical for A2/Kb mice. Therefore, CTLs to CMV-IE1 and to CMV-pp65mII are present and can be stimulated simultaneously after prime and boost. In addition, low titers of rAAV inoculum are capable of significantly boosting existing memory T cells.

### Example 9

### Detection of Humoral Response to CMV-IE1 and CMV-pp65mII in Immunized A2/Kb and HHDII Mice

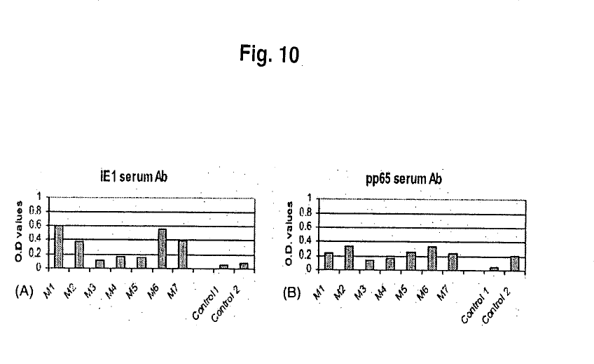

Sera from four A2/Kb and three HHDII mice, immunized using the same regimen as above in Example 8 (50 μg pcDNAintIE1, pcDNAintpp65mII and pcDNAintgm-CSF intramuscularly followed by inoculation of rAAV-IE1 and pp65mII (3×10⁵ IU/mouse)), were collected at the time of sacrifice and analyzed by ELISA for the presence of CMV-IE1- (FIG. 10A) and CMV-pp65mII-specific (FIG. 10B) antibodies.

The ELISA was performed according to methods known in the art (Endresz et al., Vaccine 17(1):50-58, 1999) using recombinant proteins CMV-IE1, CMV-pp65mII and CMV-pp150, purified from bacterial culture with the HIS-tag purification method. Immulon II™ plates were coated with 4 μg/mL recombinant protein antigen in 50 mM carbonate buffer, pH 9.6, overnight at 4° C. The plate was blocked with PBS containing 1% BSA and 0.3% gelatin (BGP) at room temperature for up to 2 hours, then washed with PBS containing 0.05% Tween™-20 (PBST). One hundred microliters of 1:50 dilution mouse serum or a serial dilution of control antibody then was added. The plate was incubated at 37° C. for 1 hour or at 4° C. overnight. After sequential staining with biotinylated anti-mouse-IgG and extravidin-peroxidase, 3,3′,5,5′-tetramethylbenzidine (TMB) substrate was added and the reaction allowed to proceed at room temperature in the dark for 15 minutes. The reaction was stopped with 50 μL of 1N sulfuric acid and the optical density read at 450 nm.

The background values obtained using control CMV-pp150-coated plates were subtracted. FIGS. 10A and 10B show the optical density values obtained. Mice M1, M2, M6 and M7 made detectable levels of antibody to CMV-IE1 and CMV-pp65, showing that mice responding to the prime-boost regimen also exhibited a humoral response to CMV-IE1 and CMV-pp65 genes.

### Example 10

### Detection of Tetramer-Positive Splenocytes in Immunized Mice

Tetramers representing the HLA A*0201 molecule (Tet) and folded with either pp65-495 (NLVPMVATV; SEQ ID NO:10) or IE1-297 (TMYGGISLL; SEQ ID NO:2) were used to detect CTLs in splenocytes of immunized mice. Mouse M7 produced antibody to CMV-pp65 and CMV-IE1, as shown in FIG. 10, and contained CTLs that were highly immunoreactive to both CMV genes by chromium release assay as well.

Tetramers were prepared essentially as described by Lacey et al., Transplantation 74(5):722-732, 2002. The tetramer reagents were folded using CMV peptides specific for the pp65 protein (SEQ ID NO:10) and for the CMV-IE1 protein (SEQ ID NO:2), and conjugated with streptavidin-allophycocyanin. One microgram of tetramer reagent was incubated for 1 hour on ice in the dark with 3×10⁵ splenocytes. After washing with PBS containing 0.5% BSA, the cells were labeled with FITC-conjugated murine DC8 antibody (Pharmingen™) for 20 minutes on ice in the dark, washed, resuspended in sheath fluid (sterile PBS) and analyzed with a FACScaliber™ flowcytometer. The lymphocyte gate was set based on forward and side scatter and a minimum of 50,000 events were captured.

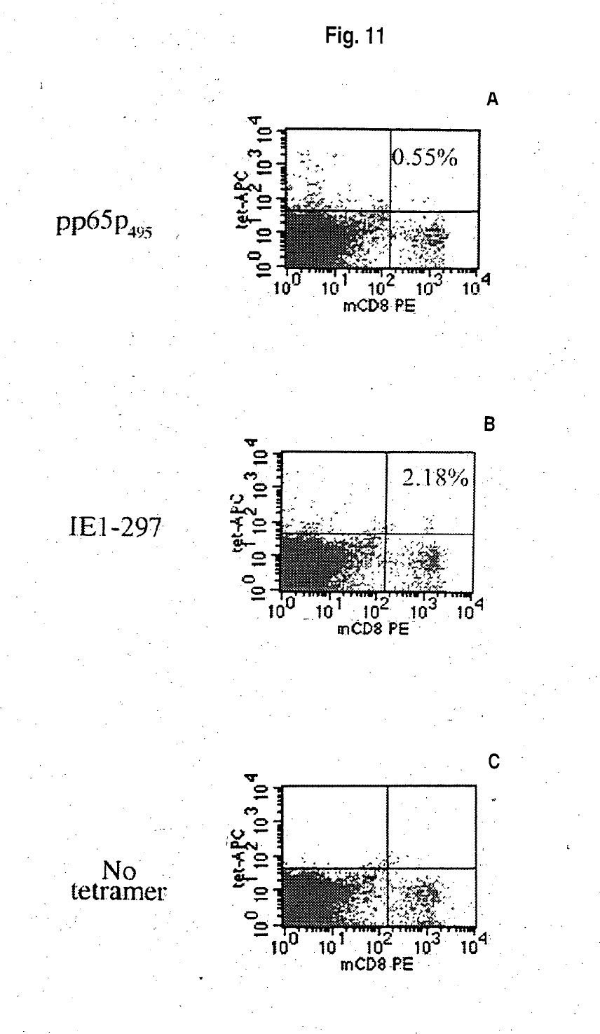

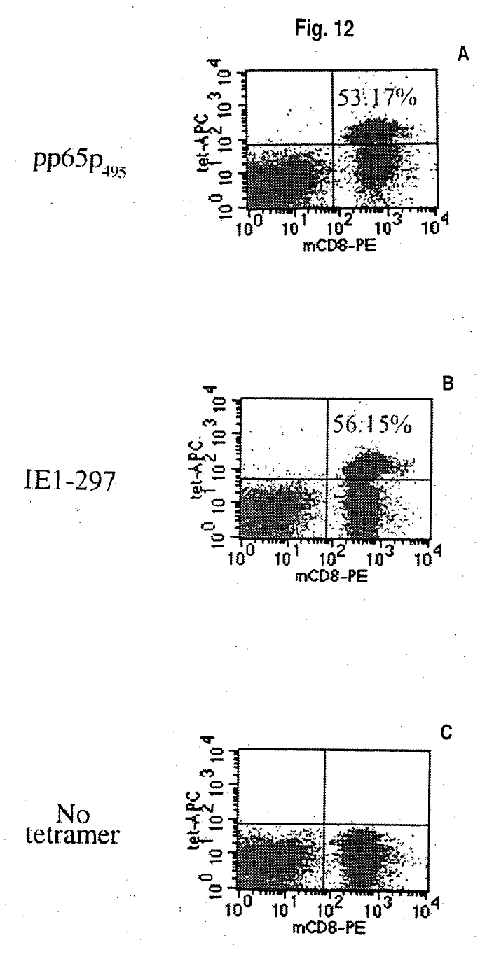

The splenocytes of M7 (same cells as in Example 8) were examined ex vivo using a tetramer binding assay to CMV-IE1 and to CMV-pp65. M7 splenocytes were incubated at time of harvest and after 6 days of stimulation with Tetpp65-495 or TetIE1-297. Percent background levels (0.02%-0.1% were subtracted from the reported percent tetramer binding cells. These M7 splenocytes showed 2.18% and 0.55% positive CD8 cells, respectively. This effect was amplified with a 6-day blast/peptide stimulation as shown in FIGS. 11 and 12 (11A-11C: ex vivo; 12A-12C: 6 days stimulation). FIGS. 11A and 12A show data obtained with SEQ ID NO:10; FIGS. 11B and 12B show data obtained with SEQ ID NO:2; FIGS. 11C and 12C show data obtained with no tetramer. The number of CD8+/TetIE-297+ cells increased to 56% of total CD8+ cells and the CD8+/Tetpp65-495+ cells to 53%. Therefore, murine transgenic CTLs could be detected using HLA A*0201 tetramers made to recognize human CTLs.

### Example 11

### Immune Responses in Human Hematopoietic Cell Transplantation Patients

Five CMV seropositive HLA A2 human subjects showing CMV reactivation within 100 days post hematopoietic cell transplantation (HCT) and 6 CMV seropositive subjects without CMV reactivation were followed at days 40, 90, 120, 150, 180 and 360 post HCT. The intracellular IFN-γ response (ICC) to CMVpp65p₄₉₅₋₅₀₃, CMV-IE1-256, IE1-297 and IE1-316 and the CD8 MHC/peptide binding (tetramer assay) using CMVpp65ₚ₄₉₅₋₅₀₃, IE1-297 and IE1-316 were analyzed in two groups. Of 31 samples in the CMV reactivation group, as detected by either PCR or shell vial assay, the frequency of positive ICC responses for each peptide was 26, 15, 14 and 17 respectively, and in 32 samples from the no-reactivation group, it was 22, 5, 4 and 10 respectively. The pp65 and IE1 tetramer binding did not significantly differ between the two groups. The ICC response to individual IE1 peptides varied over time within the same subjects and was lower in the no reactivation group.

### Example 12

### Patients and HCT Protocols

Eleven HLA A*0201 HCT recipients, at risk for CMV infection because of donor and/or recipient CMV-seropositivity, were enrolled in this study. See Table VI. The subjects included related sibling donors and recipients of allogeneic HCT for hematologic malignancies as well as HCT recipients of matched unrelated donor (MUD).

Peripheral blood mononuclear cells (PBMC) from heparin-treated whole blood were isolated using Histopaque™-1077 density gradients, washed with 1× phosphate-buffered saline (PBS) and cryopreserved in aliquots of 3×10⁶ to 5×10⁶ cells/mL in 90% fetal bovine serum (FBS) and 10% dimethylsulfoxide. The plasma was collected by centrifugation, filtered through 0.45 μm Acrodisc™ filters and stored at −20° C. until DNA extraction.

Donor samples (except for MUD subjects) were drawn before administration of granulocyte-colony stimulating factor (G-CSF) and later at the time of harvest. Recipient blood samples were collected at day 40, 90, 120, 150, 180 and 360 post-transplant for ICC and tetramer binding assays, and the CMV reactivation was monitored starting at day 21 post-HCT and twice weekly until day 100 using a shell vial assay and quantitative PCR.

Quantitative PCR was performed with DNA extracted from 200 μL plasma samples using the QIamp™ DNA Blood mini-kit (Qiagen™) and resuspended in 200 μL elution buffer. A gB CMV DNA sequence was amplified using the forward primer: 5=CTGGCCAGGCCCAAGAC (SEQ ID NO:16), the reverse primer: 5=CGGCCATTTACAACAAACCG (SEQ ID NO:17) and 100 μM probe 5=FAM-CCCATGAAACGCGCGGCA-TAMRA (Applied Biosystems™; SEQ ID NO:18) in a 30 μL reaction that contained the Taqman Universal PCR Mix™ and 10 μL of extracted DNA. The PCR cycles were set according to the manufacturer's protocol: 2 minutes at 50° C., 10 minutes at 95° C., followed by 40 cycles of 15 seconds at 95° C. and 1 minute at 60° C., and the data were collected and analyzed on an ABI Prism™ 7900HT Sequence Detection System. Serial dilutions (10⁰-10⁶ genome copies) of the plasmid containing the amplified sequence (pDCMVgB) were used to create a standard curve. No PCR inhibition was detected in samples when the exo gene was introduced in the PCR mixture as described in Limaye et al., J. Infect. Dis. 183:377-382, 2001.

Subjects with CMV reactivation as determined by 1 positive shell vial sample were treated with pre-emptive ganciclovir for 6 weeks according to known methods. Placement in the “CMV Group” required at least one positive CMV blood culture or 2-consecutive positive PCR; placement in the “No CMV Group” meant that these conditions were not met despite frequent viral surveillance. All patients were evaluated at ≧90% of the scheduled viral and immunologic surveillance time points.

The HCT protocol was essentially performed as described in Bensinger et al., N. Engl. J. Med. 344:175-181, 2001, the disclosures of which are hereby incorporated by reference. Briefly, the disease-specific conditioning regimens that consisted of high-dose chemotherapy with or without total-body irradiation were administered before transplantation. The marrow was collected from the donor by standard techniques on the day of infusion. Peripheral-blood cells were collected after treatment of donor with subcutaneous G-CSF (16 μg per kg of body weight/d for 4 days). After cell infusion, methotrexate and cyclosporine were given for the prevention of graft-versus-host disease (GVHD). The grade of GVHD was distributed as follows in the 2 groups: 1 subject in each group (#65 and #105) did not have GVHD, 2 subjects in the CMV Group had GVHD grade III (#54 and #93); GVHD grade≦II only occurred in 5 of the 6 subjects of the No CMV Group. The period of prednisone therapy at a dose of ≧1 mg/kg/day was somewhat longer in the CMV Group in 2 subjects with grade III GVHD. Ganciclovir was used only in the CMV Group, but there was a clinical decision not to treat subject #105, who was CMV positive by quantitative PCR only, and the overall outcome of these subjects at 1 year after HCT was the same. Therefore, GVHD grade III and CMV reactivation were the main clinical parameters that differentiated the groups.

TABLE VI
Allogenic Stem Cell Transplant Subject Demographics.

| | | CMV | | | | | | | | | |
|---|---|---|---|---|---|---|---|---|---|---|---|
| | | sero- | | | | | | | | | |
| | | status | Stem | | | CMV PCR | | | | | |
| Subj. | | (Donor/ | Cell | | | (genome | CMV | Antiviral | | | Clinical |
| No. | Diagnos. | Recip.) | Source | HLA | HLA | copies/mL) | BC | Treat. | GVHD Grade | Medication | Outcome |
| #53 | ALL Ph+ | D+/R+ | sibling | A0101 | A0201 | — | — | no | GVHD of the mouth: | CSA: | alive |
| | | | PBCS | | | | | | (1 m-8 m) | 0-6 months | |
| | | | | | | | | | | MMF: | |
| | | | | | | | | | | d28-6 weeks | |
| #65 | CML | D+/R− | MUD | A0201 | A1101 | — | — | no | no | | alive |
| | | | BMT | | | | | | | | |
| #76 | ALL | D+/R+ | MUD | A0201 | A6901 | — | — | no | GVDH II: skin and gut | PSE: 3 days; | expired: |
| | | | BMT | | | | | | (1 m); liver (5 m); skin | FK506 | mulit- |
| | | | | | | | | | and eyes (5 m) | (0 m-3 m); | organ |
| | | | | | | | | | | PSE (6 m-7 m); | failure |
| | | | | | | | | | | MMF (6 m-9 m) | (20 m) |
| #81 | preB- | D+/R+ | sibling | A0206 | A2401 | — | — | no | GVDH: liver (4 m) | CSA: (4-5 m); | alive |
| | ALL | | PBSC | | | | | | | MMF (4-5 m); | |
| | | | | | | | | | | PSE (4-12 m) | |
| #91 | ALL | D+/R+ | sibling | A0201 | A2601 | — | — | no | GVDH II: gut (1 m); | CSA: 0 m-1 m | alive |
| | | | PBSC | | | | | | liver (4 m) | | |
| #98 | CML | D+/R+ | sibling | A0205 | A1101 | — | — | no | GVDH: mouth (1 m); | PSE: 15 days; | alive |
| | | | PBSC | | | | | | liver (4 m) | CSA: 15 days | |
| #54 | biphenotypic | D−/R+ | MUD | A0101 | A0201 | 1416-10234 | 69 | GCV | CMV colitis; GVDH | PSE: 3 days; | alive |
| | leukemia | | BMT | | | (37) | | | III: gut (1 m); mouth | MMF: (1 m-4 m) | |
| | | | | | | | | | and eyes (10 m) | | |
| #70 | AML | D+/R+ | sibling | A0203 | A0206 | 4755-12331 | 41 | GCV | GVH II: gut (1 m); | CSA: | alive |
| | | | PBSC | | | (41) | | | stomatitis (2 m) | (0 m-15 m); | |
| | | | | | | | | | | PSE: (1 m-6 m); | |
| | | | | | | | | | | MMF: (8 m-12 m) | |
| #93 | ALL | D−/R+ | sibling | A0201 | A3001 | 202-1838 | 55 | GCV | GVDH III: gut and | CSA: (2-3 m)); | alive |
| | | | PBSC | | | (39) | | | liver (2 m) | MMF: (2-3 m); | |
| | | | | | | | | | | PSE: (2-3 m) | |
| #94 | Hodgkin = s | D+/R+ | MUD | A0201 | A0301 | — | 50 | GCV | GVDH: gut and liver | CSA: (2-3 m); | alive |
| | disease | | PBSC | | | | | | (2 m) | MMF: (2-3 m) | |
| #105 | CML | D+/R+ | sibling | A0201 | A6801 | 205 (72) | — | no | no | | alive |

### Example 13

### IFN-γ Response to CMV Peptides Stimulation in the CMV Reactivation and No CMV Groups

To evaluate the immune reactivity to CMV in HLA-A2 subjects, cryopreserved peripheral blood lymphocytes (PBL) from the subjects were stimulated with peptides derived from CMV-pp65 and CMV-IE1 proteins. Samples from each patient were thawed at 37° C., washed with cold RPMI with 10% FBS, and aliquots containing approximately 1×10⁶ PBL were stimulated with individual peptides pp65-495, IE1-256, IE1-297 and IE1-316 in separate tubes. The positive stimulation control contained phytohemagglutinin (PHA) and the negative control contained HIV peptide.

The ICC assay was adapted from Dunn et al., J. Infect. Dis. 186:15-22, 2002 for frozen cells. After 1 hour incubation at 37° C. in 5% CO₂, 1 μL (stock 5 mg/mL) of Brefeldin A, a cytokine secretion inhibitor, was added to the cells and further incubated overnight. Samples were then washed with 1×PBS and 0.5% bovine serum albumin and stained for 20 minutes in the dark with 5 μL of anti-CD8 antibody conjugated to streptavidin-phycoerythrin (CD8-PE). The cells were fixed and permeabilized for 20 minutes using the Cytofix/Cytoperm Kit™ (Pharmingen™) and stained for 30 minutes at 4° C. in the dark with 1 μL of anti-IFN-γ conjugated with streptavidin-allophycocyanin (APC). Percent CD8+IFN-γ⁺ cells as measured by ICC is reported in Table VII.

Peptides were synthesized using standard Fmoc protocols, with purification to 90% by HPLC. After purification, the peptides were dissolved in 10% DMSO/water to a concentration of 5 mM and used at a final concentration of 25 μM for T cell stimulation. The following peptides were used: IE1-256 (ILDEERDKV; SEQ ID NO:1), IE1-297 (TMYGGISLL; SEQ ID NO:2), IE1-316 (VLEETSVML; SEQ ID NO:3), pp65-495 (NLVPMVATV; SEQ ID NO:10) and HIV-468 (ILKEPVHGV; SEQ ID NO:13). The latter two peptides served as a positive and negative control, respectively.

TABLE VII
Percent Cytokine (ICC) IFN-γ+ Cells After
Stimulation with CMV pp65p495 and CMV-IE1.

| CMV reactivation | | | | | | | No CMV reactivation | | | | | |
|---|---|---|---|---|---|---|---|---|---|---|---|---|
| % CD8+/IFN-γ+ after peptide stimulation | | | | | | | % CD8+/IFN-γ+ after peptide stimulation | | | | | |
| Subj. | Days post- | | | | | | Subj. | Days post- | | | | |
| No. | HCT | pp65-495 | IE1-256 | IE1-297 | IE1-316 | PHA | No. | HCT | pp65-495 | IE1-256 | IE1-297 | IE1-316 |
| 54 | Day 40 | 0 | 0.08 | 0.01 | 0 | 0.54 | 53 | Day 40 | 0.24 | 0.1 | 0 | 0.14 |
| | Day 90 | 0.23 | 0 | 0 | 0 | 5.34 | | Day 90 | 0.06 | 0.01 | 0 | 0 |
| | Day 150 | 1.39 | 0 | 0.01 | 0.04 | 1.78 | | Day 360 | 0.03 | 0 | 0 | 0.06 |
| | Day 180 | 2.73 | 0.01 | 0 | 0 | 4.27 | 65 | Day 40 | 0 | 0 | 0.08 | 0 |
| | Day 360 | 2.35 | 0.06 | 0.05 | 0.48 | 6.91 | | Day 90 | 0.12 | 0.08 | 0 | 0.04 |
| 70(D) | preG | 0.15 | 0.11 | 0.11 | 0 | 0.24 | | Day 120 | 0 | 0 | 0 | 0 |
| | postG | 0 | 0 | 0 | 0.31 | 0.63 | | Day 150 | 0 | 0 | 0 | 0 |
| 70 | Day 40 | 0.63 | 0.1 | 0 | 0.01 | 0.64 | | Day 180 | 0.05 | 0.02 | 0.02 | 0 |
| | Day 90 | 0.87 | 0 | 0.02 | 0 | 5.09 | | Day 360 | 0 | 0.04 | 0 | 0 |
| | Day 120 | 0.68 | 0.04 | 0 | 0.1 | 4.01 | 76 | Day 90 | 0 | 0 | 0 | 0 |
| | Day 150 | 0.8 | 0.06 | 0.08 | 0.12 | 2.44 | | Day 150 | 0.21 | 0 | 0.02 | 0 |
| | Day 180 | 1.7 | 0 | 0 | 0 | 2.88 | | Day 180 | 0 | 0 | 0 | 0 |
| | Day 360 | 1.28 | 0 | 0.01 | 0.01 | 3.5 | | Day 360 | 0.06 | 0 | 0.05 | 0.15 |
| 93(D) | preG | 0.05 | 0 | 0 | 0 | 2.08 | 81 | Day 40 | 7.19 | 0 | 0 | 0.6 |
| | postG | 0 | 0 | 0 | 0 | 6.08 | | Day 90 | 7.63 | 0 | 0 | 2.28 |
| 93 | Day 40 | 2.43 | 0 | 0 | 0 | 6.32 | | Day 120 | 1.84 | 0 | 0 | 2.48 |
| | Day 120 | 7.33 | 0.04 | 0 | 0.01 | 12.46 | | Day 150 | 0.89 | 0 | 0 | 3.46 |
| | Day 150 | 6.64 | 0 | 0 | 0.03 | 21.01 | | Day 180 | 2.34 | 0 | 0 | 1.19 |
| | Day 360 | 2.99 | 0.23 | 0.13 | 0.03 | 16.5 | | Day 360 | 2.44 | 0 | 0 | 6.11 |
| 94 | Day 40 | 0.2 | 0.3 | 0.06 | 0.06 | 23.17 | 91(D) | preG | 0.05 | 0 | 0 | 0 |
| | Day 90 | 0.04 | 0.05 | 0.17 | 0.11 | 21.58 | | postG | 0.02 | 0 | 0 | 0 |
| | Day 120 | 0.03 | 0.03 | 0 | 0.06 | 14.53 | 91 | Day 40 | 0.38 | 0 | 0 | 0 |
| | Day 150 | 0 | 0 | 0 | 0 | 15.03 | | Day 90 | 0.69 | 0 | 0 | 0 |
| | Day 180 | 0.17 | 0 | 0 | 0.08 | 29.1 | | Day 120 | 1 | 0 | 0 | 0 |
| | Day 360 | 0.04 | 0.07 | 0.05 | 0.14 | 20.21 | | Day 150 | 0.73 | 0 | 0 | 0 |
| 105(D) | postG | 0.19 | 0.04 | 0 | 0.06 | 5.3 | | Day 180 | 2.09 | 0 | 0 | 0 |
| 105 | Day 40 | 0.21 | 0 | 0.04 | 0.04 | 11.56 | | Day 360 | 3.12 | 0 | 0 | 0 |
| | Day 90 | 6.32 | 0 | 0.07 | 0 | 40.76 | 98(D) | postG | 0 | 0 | 0 | 0 |
| | Day 120 | 3.82 | 0 | 0 | 0 | 29.38 | 98 | Day 40 | 0 | 0 | 0 | 0 |
| | Day 150 | 3.37 | 0.04 | 0.05 | 0 | 26.14 | | Day 90 | 0 | 0 | 0 | 0 |
| | | | | | | | | Day 120 | 0.03 | 0 | 0 | 0 |

Abbreviations:
preG = pre-granulocyte colony stimulating factor;
D = donor;
IFN-γ = gamma interferon

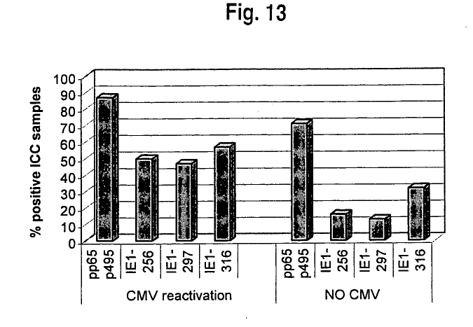

To ensure the functionality of the stimulated cells, only the samples that responded to PHA stimulation (59/61) were reported. All 5 subjects in the CMV Group responded to pp65-495 stimulation as well as to the 3 IE1 peptides. The range of response was higher using the pp65 peptide (0-10%) and lower using the IE1 peptides (0-0.48%). Every subject responded to either IE1-256, IE1-297, or IE1-316 peptides during the course of one year, but there was variability in the responses from time to time, arguing for the use of a mixture of IE1 peptides to analyze the immune response to the CMV-IE1 protein. Interestingly, 2 out of 6 subjects (#91 and #98) from the No CMV Group did not respond to IE1 peptides stimulation, although they showed IFN-γ release in the presence of pp65-495. One subject (#81), who was without detectible CMV reactivation, showed reactivity to pp65-495 and IE1-316 at levels of up to 7.63% and 6.11% respectively. Otherwise, the percentage of positive samples by IFN-γ⁺ stain was generally lower in the No CMV Group (see FIG. 13: 71%, 16%, 13% and 32% when stimulated with pp65-495, IE1-256, IE1-297 and IE1-316, respectively). In contrast, in the CMV Group, the percentages were higher for these peptides (see FIG. 13: 87%, 50%, 47% and 57%, respectively). Therefore, with regard to the cellular responses to IE1 peptides, there is immune reconstitution to CMV after HCT, and the cellular recognition is directed to multiple IE1 peptides in all subjects of the CMV Group. The No CMV Group was generally characterized by negative or low responses to IE1 peptides.

Blood samples were collected at days 40, 90, 120, 150, 180 and 360 post-HCT and tested for the kinetics of ICC/IFN-γ⁺ cells. The IFN-γ⁺ cells stimulated with IE1-256, IE1-297, and IE1-316 were reported as the sum of total number of cells×10⁵/L for the evaluation of the response to CMV-IE1 (see FIG. 14). The IFN-γ⁺ cells in the CMV Group stimulated with pp65-495 peaked at days 120 and 150 post-HCT, reaching median levels of 2.75×10⁷ cells/L. These subjects maintained peptide-responsive cells for one year (median: 3.12×10⁷ cells/L). The levels of IFN-γ⁺ cells (pp65-495) in the No CMV Group reached a peak (median: 1.8×10⁶cells/L) at day 150 and decreased to 3.0×10⁵ cells/L after 1 year (FIG. 14C). Therefore, even though the CD8⁺ CMV-pp65 reactive cells in the No CMV Group were present after transplant, the levels were not maintained and decreased nearly to the limit of detection after one year. The levels of CMV-IE1/IFN-γ⁺ responsive cells (FIGS. 14B and 14D) were a log lower than the response to CMVpp65 and did not seem to increase in number until late after HCT. The median CMV-IE1 immune response in the CMV Group was 3.8×10⁶cells/L at one year (FIG. 14B) compared to 4×10⁵ cells/L in the No CMV group (FIG. 14D).

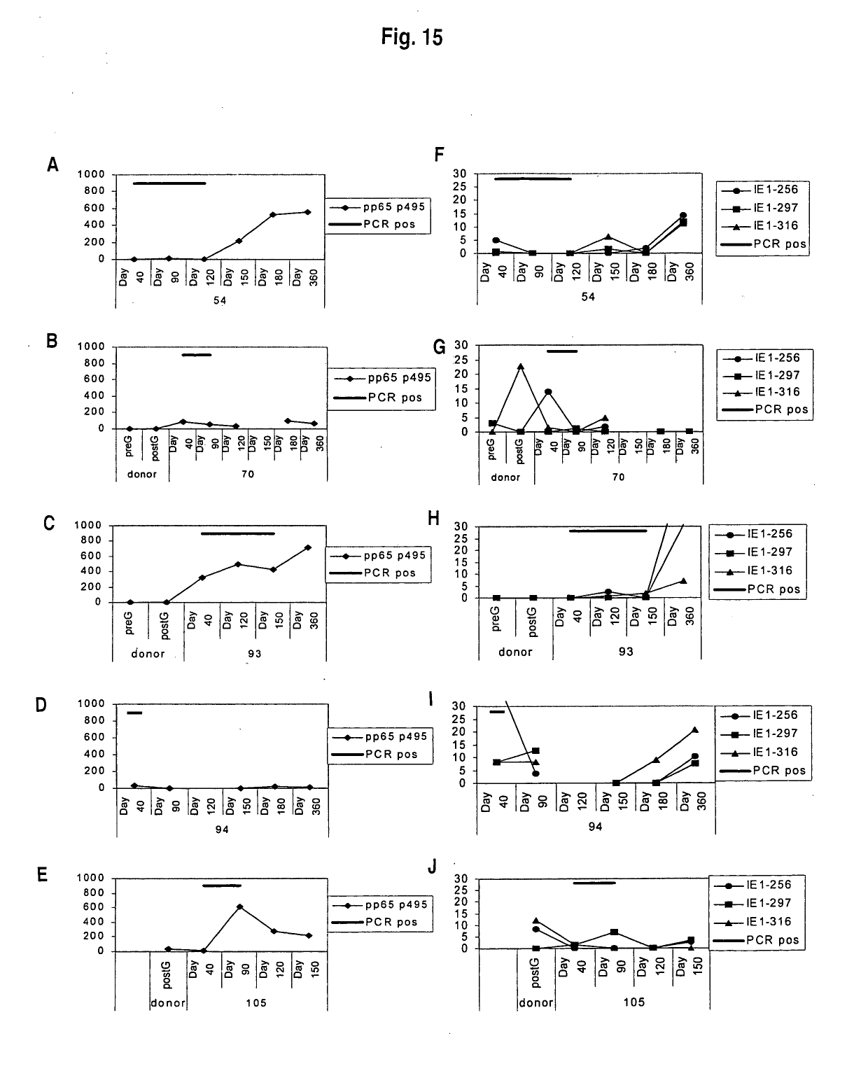

Whether the “late” appearance of CMV-IE1/IFN-γ⁺ cells was reproducibly the result of an immune response to CMV reactivation and amplification, an anticipated effect, was investigated as follows. CMV reactivation started at a median day 55 post-HCT, and as shown in FIG. 15, the appearance of the CMV/IFN-γ response to IE1 failed to demonstrate a peak response following this time of infection. Similarly, the number of IFN-γ⁺ cells stimulated with pp65-495 failed to immediately follow the occurrence of infection (FIG. 15A). Subject #105 had 3 CMV-PCR positive samples around day 72, no ganciclovir treatment and no GVHD. His ICC response to pp65-495 (6.09×10⁷ cells/L) and to IE1-297 (6.75×10⁵ cells/L) peaked at day 90. Donor cells from subject #105 responded to pp65-495, IE1-256 and IE1-316 but in the recipient of subject #105's cells, the response to IE1-316 became undetectable and then was replaced by an IE1-297 response. Recipient #94 had a CMV positive blood culture at day 50, received ganciclovir and had extensive GVHD of gut and liver. While his ICC response to pp65-495 was low for that peptide (2×10⁶ cells/L), his response to all IE1 peptides was noticeable (from 3.75×10⁵ to 4×10⁶ cells/L). The reactivation for all 3 IE1 peptides by ICC were found at day 360 in recipient #94 (MUD transplant, extensive GVHD), as well as in recipient #93 (GVHD grade III, positive PCR) and recipient #54 (MUD transplant, GVHD grade III, positive PCR). The simultaneous IE1 peptide response within the same sample was not observed in the No CMV group.

Concerning recipient #70, the positive PCR samples spanning over a period of days 41-58 post-HCT, ganciclovir treatment and GVHD grade II, was followed by low CMV-pp65 ICC response but high CMV-IE1 response. The CMV-IE1 response was present in the donor cells and decreased in the recipient during the prolonged course of prednisone. The ICC response to pp65-495 thus was independent from increased levels to CMV-IE1 peptides. PCR positivity, i.e. CMV reactivation or replication, does not directly drive the presence of CMV-IE1 stimulated ICC cells, but a combination of factors such as MUD transplantation, GVHD grade with the resulting immunosuppressive regimen may increase the number of cells reactive to all 3 IE1 peptides.

### Example 14

### Assessment of the Immune Response by Tetramer Binding Assay

HLA A*0201 tetramers, labeled with the APC molecule, were prepared with pp65-495, IE1-297 and IE1-316 peptides. Peptides were synthesized as described in Example 13. HLA A2 tetramers were prepared according to known methods using individual CMV peptides to fold the HLA A2 heavy chain and β-2 microglobulin. These then were biotinylated and conjugated with Streptavidin-allophycocyanin (APC). The samples from each patient were thawed at 37° C., washed with 1×PBS containing 0.5% BSA and transferred into polystyrene round bottom FACS tubes. Aliquots were then individually labeled with 0.5 μg to 1 μg of tetpp65-495, tetIE1-297 and tetIE1-316, and incubated for 1 hour on ice in the dark. The samples were washed and stained with 5 μL of anti-CD8 antibody conjugated to streptavidin-PE and incubated for 20 minutes, washed again and analyzed by FACS.

For FACS, patient cell samples were washed twice with 1× Cytofix™ Wash Solution for analysis by fluorescence-activated cell sorting using a FACScalibur™ instrument. The lymphocytes were gated based on forward and side scatter. A minimum of 50,000 events were analyzed per sample. The reported data are the values obtained after subtraction of background levels acquired with HIV peptide stimulation (which ranged from 0.0% to 0.5%). Typically, the stimulation of 3 CMV seronegative donors were negative upon CMV peptide stimulation. However, values of up to 0.05% have been detected in CMV seronegative “A2” 93 (D) preg (0.05% with pp65-495, see Table VII). Therefore, data below 0.05% should be interpreted with caution.

The binding of tetramers was evaluated on the same samples tested for IFN-γ ICC above and reported in Table VIII as percent CD8+ cells. The CMV reactivation group was characterized by an increase in tetpp65-495 binding during the course of transplant in all recipients except #94, similar to the ICC data of Table VII. The range of % CD8⁺/tet⁺ cells was 0-11.85% for pp65-495, 0-0.24% for IE1-297 and 0-3.66% for IE1-316. The No CMV group did not differ, showing ranges of % CD8⁺/tet⁺ cells spanning from 0-10.18% for pp65-495, 0-0.96% for IE1-297 and 0-4.09% for IE1-316. However, when reactive CD8 cell count is analyzed during hematopoietic reconstitution (see FIG. 16), the CMV reactivation group was defined by high levels of CD8⁺/tetpp65-495 cells that appeared at day 120 post-HCT (median: 6.36×10⁷ cells/L) and was maintained through the year (median: 8.23×10⁷ cells/L). See FIG. 16A.

In contrast, the number of cells binding to CD8+/tet-IE1-297 and CD8+/tet IE1-316 expressed as a sum steadily increased to a median of 3.7×10⁶ cells/L at day 360. See FIG. 16B. The median levels of CD8+/tetpp65-495 and CD8+/tet-IE1 at day 40 post-HCT in the CMV reactivation group were similar (median 5×10⁶ cells/L and 7.4×10⁶cells/L respectively), suggesting that these cells may still have been immune cells transferred from the donor. However, upon CMV reactivation, the immune cells specific for CMV-pp65 increased to 7.58×10⁷ cells/L by day 150 whereas the cells specific to CMV-IE1 decreased to 1.8×10⁶ cells/L. Therefore, there is no correlation between the two CMV specific genes regarding the expansion of immune cells during CMV reactivation. The No CMV group developed a peak of CD8+/tetpp65-495 cells at a later time (median: 2.28×10⁷ cells/L at day 180), which decreased to 4×10⁵ cells/L by 1 year. See FIG. 16C. The levels of CD8+/tetIE1 were the same in both groups (see FIGS. 16B and 16D) except for day 40 in the CMV reactivation group as mentioned above. The null value for tetramer binding was much less frequent when compared to the ICC assay, especially in the No CMV group. The tetramer binding data therefore indicate that the CD8⁺ immune cells to CMV are present in reasonably high quantity in HCT subjects during hematopoietic reconstitution.

TABLE VIII
Percent CD8+ Tetramer A2+ Cells Specific for
CMVpp65 and IE1 Peptide.

| CMV reactivation | | | | | No CMV reactivation | | | | |
|---|---|---|---|---|---|---|---|---|---|
| % CD8 tetramer binding | | | | | % CD8 tetramer binding | | | | |
| Subj. | Days post- | | | | Subj. | Days post- | | | |
| No. | HCT | pp65-495 | IE1-297 | IE1-316 | No. | HCT | pp65-495 | IE1-297 | IE1-316 |
| 54 | Day 40 | 0.28 | 0.17 | 0.75 | 53 | Day 40 | 0.32 | 0.96 | 4.09 |
| | Day 120 | 4.17 | 0 | 2.66 | | Day 90 | 0.07 | 0.19 | 0.41 |
| | Day 150 | 5.66 | 0.05 | 0.24 | | Day 360 | 0 | 0 | 0.08 |
| | Day 180 | 4.69 | 0.01 | 0.05 | 65 | Day 40 | 0.16 | 0.06 | 0.16 |
| | Day 360 | 3.49 | 0.12 | 1.36 | | Day 90 | 0.11 | 0.04 | 0.41 |
| 70(D) | preG | 0.39 | 0.9 | 3.15 | | Day 150 | 0.02 | 0 | 0.07 |
| | postG | 0.02 | 0 | 0.02 | | Day 360 | 0.1 | 0.07 | 0.13 |
| 70 | Day 40 | 1.48 | 0.05 | 0.61 | 76 | Day 90 | 0.17 | 0.16 | 0.77 |
| | Day 90 | 0.86 | 0.04 | 0.13 | | Day 360 | 0.18 | 0.16 | 0.77 |
| | Day 360 | 1.78 | 0 | 0.06 | 81 | Day 150 | 0.11 | 0.11 | 0.98 |
| 93(D) | preG | 0.08 | 0 | 0.08 | | Day 40 | 6.4 | 0.04 | 0.06 |
| | postG | 0.12 | 0.17 | 0.26 | | Day 90 | 8.49 | 0.02 | 0.02 |
| 93 | Day 120 | 9.36 | 0 | 0.01 | | Day 120 | 6.18 | 0.05 | 0.21 |
| | Day 150 | 11.85 | 0.04 | 0.09 | | Day 150 | 4.6 | 0.03 | 0.11 |
| | Day 360 | 4.73 | 0.01 | 0.04 | | Day 180 | 4.54 | 0.03 | 0.21 |
| 94 | Day 40 | 0.22 | 0.24 | 3.66 | | Day 360 | 10.18 | 0.09 | 0.23 |
| | Day 90 | 0.41 | 0.11 | 0.56 | 91(D) | preG | 0.14 | 0.08 | 0.34 |
| | Day 120 | 0.01 | 0.03 | 0.22 | | postG | 0.12 | 0 | 0.23 |
| | Day 150 | 0.07 | 0.02 | 0.08 | 91 | Day 40 | 1.25 | 0.01 | 0.13 |
| | Day 180 | 0.08 | 0.03 | 0.19 | | Day 90 | 0.57 | 0.13 | 0.06 |
| | Day 360 | 0.12 | 0 | 0.25 | | Day 120 | 0.35 | 0.15 | 0.21 |
| 105(D) | postG | 0 | 0 | 0.19 | | Day 150 | 0.7 | 0.3 | 0.44 |
| 105 | Day 40 | 0.14 | 0.02 | 0.02 | | Day 180 | 1.94 | 0 | 0.11 |
| | Day 90 | 4.79 | 0 | 0.01 | | Day 360 | 4.39 | 0.03 | 0.42 |
| | Day 120 | 4.36 | 0.04 | 0.21 | 98 | postG | 0.03 | 0.06 | 0.21 |
| | | | | | 98(D) | Day 40 | 0.03 | 0.07 | 0.13 |
| | | | | | | Day 90 | 0 | 0.04 | 0.08 |
| | | | | | | Day 120 | 0.02 | 0.02 | 0.09 |

Abbreviations:
preG = pre-granulocyte colony stimulating factor;
D = donor

### References

The following references are hereby incorporated by reference in their entirety.
1. Adler et al., “Immunity induced by primary human cytomegalovirus infection protects against secondary infection among women of childbearing age.” J. Infect. Dis. 171:26-32, 1995.
2. Ahn et al., “The ER-luminal domain of the HCMV glycoprotein US6 inhibits peptide translocation by TAP.” Immunity 6:613-621, 1997.
3. Akrigg et al., “The structure of the major immediate early gene of human cytomegalovirus strain AD169.” Virus Res. 2(2):107-21, 1985.
4. Aubert et al., “Cytomegalovirus-specific cellular immune responses and viremia in recipients of allogeneic stem cell transplants.” J. Infect. Dis. 184:955-963, 2001.
5. Barel et al., “Amino acid composition of alphal/alpha2 domains and cytoplasmic tail of MHC class I molecules determine their susceptibility to human cytomegalovirus US11-mediated down-regulation.” Eur. J. Immunol. 33:1707-1716, 2003.
6. BenMohamed et al., “Induction of CTL response by a minimal epitope vaccine in HLA A *0201/DRl transgenic mice: dependence on HLA class II restricted T(H) response.” Hum. Immunol. 61: 764-779, 2000.
7. Bensinger et al., “Transplantation of bone marrow as compared with peripheral-blood cells from HLA-identical relatives in patients with hematologic cancers.” N. Engl. J. Med. 344:175-181, 2001.
8. Berencsi et al., “Murine cytotoxic T cell response specific for human cytomegalovirus glycoprotein B (gB) induced by adenovirus and vaccinia virus recombinants expressing gB.” J. Gen. Virol. 174: 2507-2512, 1993.
9. Boppana et al., “Recognition of human cytomegalovirus gene products by HCMV-specific cytotoxic T cells.” Virology 222:293-296, 1996.
10. Chatterjee et al., “Dual-target inhibition of HN-1 in vitro by means of an adeno-associated virus antisease vector.” Science 258:1485-1488, 1992.
11. Chen et al., “HLA tetramers and anti-CMV immune responses: from epitope to immunotherapy.” Cytotherapy 4:41-48, 2002.
12. Cheevers et al., “Prime-boost vaccination with plasmid DNA encoding caprine-arthritis encephalitis lentivirus env and virus SU suppresses challenge virus and development of arthritis.” Virology 306(1):116-25, 2003.
13. Chou et al., “Effect of interstrain variation on diagnostic DNA amplification of the cytomegalovirus major immediate-early gene region.” J. Clin. Microbiol. 30:2307-2310, 1992.
14. Cwynarski et al., “Direct visualization of cytomegalovirus-specific T-cell reconstitution after allogeneic stem cell transplantation.” Blood 97:1232-1240, 2001.
15. Del Val et al., “Protection against lethal cytomegalovirus infection by a recombinant vaccine containing a single nonameric T-cell epitope.” J. Virol. 65: 3641-3646, 1991.
16. Diamond et al., “Development of a candidate HLA A*0201 restricted peptide-based vaccine against human cytomegalovirus infection.” Blood 90: 1751-1767, 1997.
17. Dunn et al., “Dynamics of CD4 and CD8 T cell responses to cytomegalovirus in healthy human donors.” J. Infect. Dis. 186:15-22, 2002.
18. Elkington et al., “Ex vivo profiling of CD8+-T-cell responses to human cytomegalovirus reveals broad and multispecific reactivities in healthy virus carriers.” J. Virol. 77:5226-5240, 2003.
19. Endresz et al., “Optimization of DNA immunization against human cytomegalovirus.” Vaccine 19:3972-3980, 2001.
20. Engelhard et al., “Influenza A-specific, HLA-A2.1-restricted cytotoxic T lymphocytes from HLA-A2.1 transgenic mice recognize fragments of the Ml protein.” J. Immunol. 146: 1226-1232, 1991.
21. Engstrand et al., “Characterization of CMVpp65-specific CD8+ T lymphocytes using MHC tetramers in kidney transplant patients and healthy participants.” Transplantation 69:2243-2250, 2002.
22. Falk et al., “Consensus motifs and peptide ligands of MHC class I molecules.” Semin. Immunol. 15:81-94, 1993.
23. Firat et al., “H-2 class 1 knockout HLA-A2. 1-transgenic mice: a versatile animal model for preclinical evaluation of antitumor immunotherapeutic strategies.” Eur. J. Immunol. 29(10):3112-21, 1999.
24. Foster et al., “Cytomegalovirus-specific CD4+ and CD8+ T-cells follow a similar reconstitution pattern after allogeneic stem cell transplantation.” Biol. Blood Marrow Transplant. 8:501-511, 2002.
25. Frankenberg et al., “Identification of a conserved HLA-A2-restricted decapeptide from the IE1 protein (pUL123) of human cytomegalovirus.” Virology 295:208-216, 2002.
26. Gallez-Hawkins et al., “Kinase-deficient CMVpp65 triggers a CMVpp65 specific T-cell immune response in HLA-A*0201.Kb transgenic mice after DNA immunization.” Scand. J. lmmunol. 55:592-598, 2002.
27. Gallez-Hawkins et al., “Use of transgenic HLA A*0201/Kb and HHD II mice to evaluate frequency of cytomegalovirus IE1-derived peptide usage in eliciting human CD8 cytokine response.” J. Virol. 77:4457-4462, 2003.
28. Gandhi et al., “Late diversification in the clonal composition of human cytomegalovirus-specific CD8+ T cells following allogeneic hemopoietic stem cell transplantation.” Blood 102:3427-3438, 2003.
29. Gibson et al., “Human cytomegalovirus proteins pp65 and immediate early protein 1 are common targets for CD8+ T cell responses in children with congenital or postnatal human cytomegalovirus infection.” J. immunol. 172:2256-2264, 2004.
30. Gratama et al., “Tetramer-based quantification of cytomegalovirus (CMV)-specific CD8+ T lymphocytes in T-cell-depleted stem cell grafts and after transplantation may identify patients at risk for progressive CMV infection.” Blood 98:1358-1364, 2001.
31. Gratama and Cornelissen, “Clinical utility of tetramer-based immune monitoring in allogeneic stem cell transplantation.” BioDrugs 17:325-338, 2003.
32. Gricks et al., “Somatically mutated regions of immunoglobulin on human B-cell lymphomas code for peptides that bind to autologous major histocompatibility complex class I, providing a potential target for cytotoxic T cells.” Cancer Res. 61:5145-5152, 2001.
33. Gyulai et al., “Cytotoxic T lymphocyte (CTL) responses to human cytomegalovirus pp65, IE1-Exon4, gB, pp150, and pp28 in healthy individuals: reevaluation of prevalence of IE1-specific CTLs.” J. Infect. Dis. 181:1537-1546, 2000.
34. He et al., “Direct functional analysis of epitope-specific CD8+ T cells in peripheral blood.” Viral Immunol. 14:59-69, 2001.
35. Hebart et al., “A CTL epitope from human cytomegalovirus IE1 defined by combining prediction of HLA binding and proteasomal processing is the target of dominant immune responses in patients after allogeneic stem cell transplantation.” Exp. Hematol. 31:966-973, 2003.
36. Herweijer et al., “Progress and prospects: naked DNA gene transfer and therapy.” Gene Ther. 10(6):453-8, 2003.
37. Kern et al., “Analysis of CD8 T cell reactivity to cytomegalovirus using protein-spanning pools of overlapping pentadecapeptides.” Eur. J. Immunol. 130:1676-1682, 2000.
38. Kern et al., “Target structures of the CD8(+)-T-cell response to human cytomegalovirus: the 72-kilodalton major immediate-early protein revisited.” J. Virol. 173:8179-8184, 1999.
39. Khan et al., “Comparative analysis of CD8⁺ T cell responses against human cytomegalovirus proteins pp65 and immediate early 1 shows similarities in precursor frequency, oligoclonality, and phenotype.” J. Infect. Dis. 185:1025-1034, 2002.
40. Komatsu et al., “Population analysis of antiviral T cell responses using MHC class I-peptide tetramers.” Clin. Exp. Immunol. 134:9-12, 2003.
41. Lacey et al., “Assessment of cellular immunity to human cytomegalovirus in recipients of allogeneic stem cell transplants.” Biol. Blood Marrow Transplant. 10:433-447, 2004.
42. Lacey et al., “Characterization of cytotoxic function of CMV-pp65-specific CD8+ T-lymphocytes indentified by HLA tetramers in recipients and donors of stem-cell transplants.” Transplantation 74(5):722-732, 2002.
43. La Rosa et al., “Preclinical development of an adjuvant-free peptide vaccine with activity against CMV pp65 in HLA transgenic mice.” Blood 100:3681-3689, 2002.
44. Limaye et al., “Cytomegalovirus (CMV) DNA load in plasma for the diagnosis of CMV disease before engraftment in hematopoietic stem-cell transplant recipients.” J. Infect. Dis. 183:377-382, 2001.
45. Miller et al., “Human cytomegalovirus inhibition of major histocompatibility complex transcription and interferon signal transduction.” Curr. Top. Microbiol. Immunol. 269:153-170, 2002.
46. Morello et al., “Development of a vaccine against murine cytomegalovirus (MCMV), consisting of plasmid DNA and formalin-inactivated MCMV, that provides long-term, complete protection against viral replication.” J. Virol. 176:4822-4835, 2002.
47. Newberg et al., “Species specificity in the interaction of CD8 with the alpha 3 domain of MHC class I molecules.” J. Immunol. 149:136-142, 1992.
48. Ouyang et al., “Large numbers of dysfunctional CD8+ T lymphocytes bearing receptors for a single dominant CMV epitope in the very old.” J. Clin. Immunol. 23:247-257, 2003.
49. Pancholi et al., “DNA-immunization with hepatitis C virus (HCV) polycistronic genes or immunization by HCV DNA priming-recombinant canarypox virus bossting induces immune responses and protection from recombinant HCV-vaccinia virus infection in HLA-A2.1-transgenic mice.” J. Virol. 77(1):382-90, 2003.
50. Pande et al., “Direct DNA immunization of mice with plasmid DNA encoding the tegument protein pp65 (PpUL83) of human cytomegalovirus induces high levels of circulating antibody to the encoded protein.” Scand. J. Infect. Dis. Suppl. 99:117-120, 1995.
51. Parham et al., “Partial purification and some properties of BB7.2. A cytotoxic monoclonal antibody with specificity for HLA-A2 and a variant of HLA-A28.” Hum. Immunol. 3:277-299, 1981.
52. Pepperl et al., “Dense bodies of human cytomegalovirus induce both humoral and cellular immune responses in the absence of viral gene expression.” J. Virol. 74(13): 6132-6146, 2000.
53. Plotkin et al., “Vaccines for varicella-zoster virus and cytomegalovirus: recent progress.” Science 265:1383-1385, 1994.
54. Price et al., “B-cell activation following murine cytomegalovirus infection: implications for autoimmunity.” Immunology 78:14-21, 1993.
55. Prod'homme et al., “Modulation of HLA-A*0201-restricted T cell responses by natural polymorphism in the IE1(315-324) epitope of human cytomegalovirus.” J. Immunol. 170:2030-2036, 2003.
56. Rammensee et al., “SYFPEITHI: database for MHC ligands and peptide motifs.” Immunogenetics 50:213-219, 1999.
57. Retiere et al., “A polymorphism in the major immediate-early gene delineates groups among cytomegalovirus clinical isolates.” Virus. Res. 57:43-51, 1998.
58. Retiere et al., “Generation of cytomegalovirus-specific human T-lymphocyte clones by using autologous B-lymphoblastoid cells with stable expression ofpp65 or IE1 proteins: a tool to study the fine specificity of the antiviral response.” J. Virol. 74:3948-3952, 2000.
59. Samulski et al., “Helper-free stocks of recombinant adeno-associated viruses: normal integration does not require viral gene expression.” J. Virol. 63(9):3822-8, 1989.
60. Shiver et al., “Replication-incompetent adenoviral vaccine vector elicits effective anti-immunodeficiency-virus immunity.” Nature 415(6869):331-5, 2002.
61. Szmania et al., “Isolation and expansion of cytomegalovirus-specific cytotoxic T lymphocytes to clinical scale from a single blood draw using dendritic cells and HLA-tetramers.” Blood 98:505-512, 2001.
62. Thompson et al., “Stable incorporation of a bacterial gene into adult rat skeletal muscle in vivo.” Am. J. Physiol. 258:C578-C581, 1990.
63. Villacres et al., “Maintenance of CD8(+)T-cell memory following infection with recombinant sindbis and vaccinia viruses.” Virology 270:54-64, 2000.
64. Villacres et al., “Human immunodeficiency virus-infected patients receiving highly active antiretroviral therapy maintain activated CD8+ T cell subsets as a strong adaptive immune response to cytomegalovirus.” J. Infect. Dis. 184:256-267, 2001.
65. Villacres et al., “Relevance of peptide avidity to the T cell receptor for cytomegalovirus-specific ex vivo CD8 T cell cytotoxicity.” J. Infect. Dis. 188:908-918, 2003.
66. Vitiello et al., “Analysis of the HLA-restricted influenza-specific cytotoxic T lymphocyte response in transgenic mice carrying a chimeric human-mouse class I major histocompatibility complex.” J. Exp. Med. 173:1007-1015, 1991.
67. Wiertz et al., “Cytomegaloviruses use multiple mechanisms to elude the host immune response.” Immunol. Lett. 57:213-216, 1997.
68. Wills et al., “The human cytotoxic T-lymphocyte (CTL) response to cytomegalovirus is dominated by structural protein pp65: frequency, specificity, and T-cell receptor usage of pp65-specific CTL.” J. Virol. 70:7569-7579, 1996.
69. Wins et al., “The human cytotoxic T-lymphocyte (CTL) response to cytomegalovirus is dominated by structural protein pp65: frequency, specificity, and T-cell receptor usage of pp65-specific CTL.” J. Virol. 70:7569-7579, 1996.
70. Yao et al., “Site-directed mutation in a conserved kinase domain of human cytomegalovirus-pp65 with preservation of cytotoxic T lymphocyte targeting.” Vaccine 19(13-14):1628-35, 2001.
71. Zaia et al., “Preemptive ganciclovir administration based solely on asymptomatic pulmonary cytomegalovirus infection in allogeneic bone marrow transplant recipients: long-term follow-up.” Biol. Blood Marrow Transplant. 1:88-93, 1995.
72. Zaia et al., “Infrequent occurrence of natural mutations in the pp65(495-503) epitope sequence presented by the HLA A*0201 allele among human cytomegalovirus isolates.” J. Virol. 75:2472-2474, 2001.

1. A CMV peptide composition which comprises a peptide selected from the group consisting of SEQ ID NOS: 1, 2 and 3.

2. A peptide selected from the group consisting of SEQ ID NOS: 1, 2 and 3.

3. A CMV peptide composition of claim 1 which is a vaccine composition.

4. A CMV peptide composition of claim 1 which is a diagnostic reagent.

5. A vaccine composition of claim 3 which comprises an antigen presenting cell.

6. A DNA vaccine that encodes a peptide selected from the group consisting of SEQ ID NOS: 1, 2 and 3.

7. A method of stimulating the production of CMV-specific cytotoxic T lymphocytes in a patient in need thereof which comprises administering to said patient a vaccine composition of claim 3.

8. A method of stimulating the production of CMV-specific cytotoxic T lymphocytes in a patient in need thereof which comprises administering to said patient a vaccine composition of claim 5.

9. A method of stimulating the production of CMV-specific cytotoxic T lymphocytes in a patient in need thereof which comprises administering to said patient a vaccine composition of claim 6.

10. A method of diagnosing the presence of CMV-specific cytotoxic T lymphocytes in a patient sample containing cytotoxic T lymphocytes which comprises contacting said patient sample in vitro with a diagnostic reagent of claim 4.

11. A method of claim 10 wherein said reagent is an antigen presenting cell.


SEQUENCE LISTING (18 sequences)

SEQ ID NO: 1 - protein, 9 aa, Human cytomegalovirus
ILDEERDKV

SEQ ID NO: 2 - protein, 9 aa, Human cytomegalovirus
TMYGGISLL

SEQ ID NO: 3 - protein, 9 aa, Human cytomegalovirus
VLEETSVML

SEQ ID NO: 4 - protein, 9 aa, Human cytomegalovirus
VLAELVKQI

SEQ ID NO: 5 - protein, 9 aa, Human cytomegalovirus
SLLSEFCRV

SEQ ID NO: 6 - protein, 9 aa, Human cytomegalovirus
LLSEFCRVL

SEQ ID NO: 7 - protein, 9 aa, Human cytomegalovirus
YVLEETSVM

SEQ ID NO: 8 - protein, 10 aa, Human cytomegalovirus
YILGADPLRV

SEQ ID NO: 9 - protein, 9 aa, Human cytomegalovirus
ILGADPLRV

SEQ ID NO: 10 - protein, 9 aa, Human cytomegalovirus
NLVPMVATV

SEQ ID NO: 11 - protein, 11 aa, Human cytomegalovirus
RPHERNGFTVL

SEQ ID NO: 12 - protein, 10 aa, Human cytomegalovirus
TPRVTGGGAM

SEQ ID NO: 13 - protein, 9 aa, Human immunodeficiency virus
ILKEPVHGV

SEQ ID NO: 14 - DNA, 33 bp, Mus musculus
tatagcggccgcctcagagagaaaggctaaggt

SEQ ID NO: 15 - DNA, 30 bp, Mus musculus
gccttctgtttgctctctatcccgggatat

SEQ ID NO: 16 - DNA, 17 bp, Human cytomegalovirus
ctggccaggcccaagac

SEQ ID NO: 17 - DNA, 20 bp, Human cytomegalovirus
cggccatttacaacaaaccg

SEQ ID NO: 18 - DNA, 18 bp, Human cytomegalovirus
  misc_feature (1)..(1): labeled with reporter dye FAM
cccatgaaacgcgcggca